(19) United States
(12) Patent Application Publication
(10) Pub. No.: US 2024/0238076 A1
(43) Pub. Date: Jul. 18, 2024

(54) PROCESS FOR PRODUCING VASCULAR PROSTHESES

(71) Applicants: Medizinische Universität Wien, Wien (AT); Technische Universität Wien, Wien (AT); Ludwig Boltzmann Gesellschaft-Osterreichische Vereinigung zur Forderung von wissenschaftlichen Fors+, Wein (AT)

(72) Inventors: Stefan BAUDIS, Wien (AT); Helga BERGMEISTER, Wien (AT); Katharina EHRMANN, Wien (AT); Christian GRASL, ******* (AT); Robert LISKA, Schleinbach (AT); Bruno PODESSER, Wien (AT); Heinrich SCHIMA, Wien (AT)

(21) Appl. No.: 18/274,707

(22) PCT Filed: Jan. 28, 2022
(86) PCT No.: PCT/EP2022/052088

§ 371 Date: Feb. 9, 2024

(30) Foreign Application Priority Data
Jan. 28, 2021 (EP) 21153995.2

(51) Int. Cl. A61F 2/06; A61L 27/18; A61L 27/50; A61L 27/58; C08G 18/10; C08G 18/24; C08G 18/32; C08G 18/42; C08G 18/44; C08G 18/73; D01D 5/00; D01F 6/78

(52) U.S. Cl. CPC A61F 2/06; A61L 27/18; A61L 27/507; A61L 27/58; C08G 18/10; C08G 18/244; C08G 18/3215; C08G 18/3234; C08G 18/3246; C08G 18/325; C08G 18/4277; C08G 18/44; C08G 18/73; D01D 5/003; D01F 6/78; A61F 2240/001; D10B 2331/10; D10B 2331/12; D10B 2401/063; D10B 2401/12; D10B 2509/06

(57) ABSTRACT

A thermoplastic poly(urethane-urea) polyadduct (I) with sterically hindered urea groups used in an electrospinning method for producing vascular prostheses:

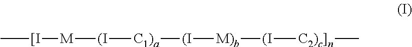

I, M, C₁ and C₂ represent bivalent radicals linked via a urethane or urea moiety;
I and C₂ represent a bivalent, saturated or unsaturated, aliphatic, alicyclic or aromatic radical with 1-20 carbon atoms derived from (I): a diisocyanate; or (C₂): from a diol, diamine or amino alcohol;
M represents a bivalent radical of an aliphatic polyether, polyester or polycarbonate derived from a macrodiol having Mₙ≥500;
C₁ represents a bivalent, saturated or unsaturated, aliphatic or alicyclic radical with 1-30 carbon atoms derived from a diamine or amino alcohol with ≥1 sterically hindered secondary amino group through removal of one N-linked hydrogen atom of the diamine or one N-linked and the O-linked hydrogen atoms of the amino alcohol.

The present invention relates to a new method for producing vascular prostheses from thermoplastic poly(urethane-urea) adducts by electrospinning.

### STATE OF THE ART

Many patients worldwide are affected by cardiovascular diseases. Occlusion of medium and small diameter vessels is the most common pathologic cause of cardiovascular diseases. Despite significant advances in interventional revascularization procedures, surgical therapy is often indicated to treat occluded blood vessels. Because of their advantageous biomechanical properties and low thrombogenicity, autologous blood vessels are the preferred substitute for the reconstruction of small-diameter blood vessels. However, in many patients this therapeutic approach fails due to insufficient vascular quality of the autologous graft. Prosthetic vascular prostheses for small-diameter vascular replacement that are, in their long-term performance, equivalent to autologous blood vessels are currently not available. Synthetic implants have inadequate biomechanical properties and favor the formation of thrombi.

Biodegradable vascular prostheses, which are vitalized in situ by the host with vessel-specific host cells after implantation, are a new promising therapeutic approach. The ultimate goal of this application is the formation of a new, functional blood vessel after a complete degradation of the synthetic construct. However, it is essential for the success of the application that the biological activity of the recipient and the properties of the prosthesis are matched so that degradation and replacement by the body's own tissue are synchronized.

Small-lumen vascular prostheses made from thermoplastic polyether and polycarbonate urethanes by electrospinning gave very good results when tested in preclinical studies; see, for example, Baudis S., Ligon S. C., Seidler K., Weigel G., Grasl C., Bergmeister H., Schima H., Liska R., “Hard-block degradable thermoplastic urethaneelastomers for electrospun vascular prostheses,” J. Polym. Sci. A1 50, 1272-1280 (2012); Seidler K., Ehrmann K., Steinbauer P., Rohatschek A., Andriotis O. G., Dworak C., Koch T., Bergmeister H., Grasl C., Schima H., Thurner P. J., Liska R., Baudis S., “A structural reconsideration: limear alphatic or alicyclic hard segments for biodegradable thermoplastic polyurethanes?”, J. Polym. Sci. A1 56, 2214-2224 (2018); and Ehrmann K., Potzmann P., Dworak C., Bergmeister H., Eilenberg M., Grasl C., Koch T., Schima H., Liska R., Baudis S., “Hard Block Degradable Polycarbonate Urethanes: Promising Biomaterials for Electrospun Vascular Prostheses,” Biomacromolecules 21(2), 376-387 (2020)). These documents disclose various biodegradable thermoplastic polyurethanes (TPUs), more specifically polyether and polycarbonate urethanes, which are prepared by polyaddition of diisocyanates, macrodiols and chain extenders. The radicals of the chain extenders and diisocyanates are known as hard segments, and those of the macrodiols as soft segments. The materials disclosed in the state of the art all have good thermal-mechanical properties, are easy to handle when used for the preparation of vascular prostheses by electrospinning, and have also shown good biodegradability when used as implants in animal tests. However, for a successful clinical application, implants with further improved biomechanical properties would be desirable in order to ensure safe long-term use.

Polymers that often show quite good mechanical properties also include polyurethane/polyurea thermoplasts, which are usually prepared by polyaddition of diols and diamines with diisocyanates and are hereinafter referred to as thermoplastic poly(urethane-urea) adducts or TPUUs (“thermoplastic polyurethane-ureas). The structure of such adducts can generally be represented by the following Formula (1):

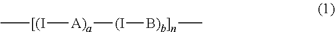

wherein I, A and B each represent bivalent radicals derived from a diisocyanate (I), a diol (A) or a diamine (B) and linked to each other via a urethane or urea moiety, a and b represent the respective number of repeats of the urethane or urea units, and n represents the number of blocks comprising the two. A more detailed exemplary representation of a structure with alternating diol units A and diamine units B, i.e., with a=b=1, is the following Formula (2):

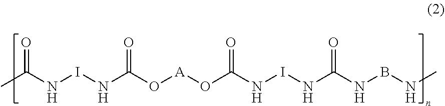

Therein, in addition, two urethane moieties, —NH—CO—O— and —O—CO—NH—, derived from the addition of a diol, HO-A-OH, to two diisocyanate molecules alternate with two urea moieties, —NH—CO—NH—, derived from the addition of a diamine, H₂N—B—NH₂, to two diisocyanates. If a and b represent numbers>1, polyurethane and polyurea segments alternate within a block.

A representation using secondary diamines for forming the urea units is somewhat more complex because, in these cases, two further radicals attached to the nitrogen atoms have to be taken into account in addition to radical B. For example, an N,N′-dimethyl derivative of the above diamine would result in a structure according to the following Formula (3):

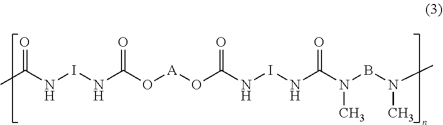

The general structure could be represented in a simplified way by referring to the bivalent radical, —NCH₃—B—NCH₃—, obtained by removing the two N-linked hydrogen atoms of the diamine as radical D, as shown in Formula (4):

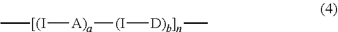

In this case, a combination of the two representations results in a structure of the following Formula (5):

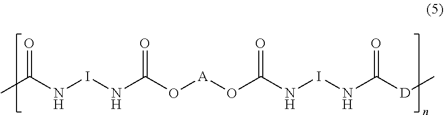

wherein the two nitrogen atoms of radical D each complete an N-methylated urea moiety.

The mechanical properties of TPUUs can be controlled within relatively large ranges by selecting suitable monomers, e.g., by providing a suitable ratio of hard and soft segments within the polymer chains. For example, EP 452,775 A2 discloses the preparation of TPUUs with increased heat resistance using 4,4′-diisocyanatodicyclohexylmethane and optionally methylated piperazine for producing polyurea hard segments, which are combined with polyurethane soft segments made from macrodiols.

In addition, it has been known for decades that, in sterically hindered urea molecules, the stability of the bond between nitrogen atoms substituted with at least one voluminous radical and the carbonyl group is instable. The dissociation of urea molecules with different bulky substituents was already described in 1974, including various combinations of isopropyl, sec-butyl, tert-butyl, 3-pentyl, cyclohexyl and isooctyl substituents on the same nitrogen atom as well as a cyclic variant using tetramethylpiperidine (Stowell, J. C., Padegimas, S. J., “Urea dissociation. Measure of steric hindrance in secondary amines”, J. Org. Chem. 39(16), 2448-2449 (1974)).

The equilibrium reaction taking place in this case can be represented, for example, as shown in the following Scheme A for a double substitution with tert-butyl:

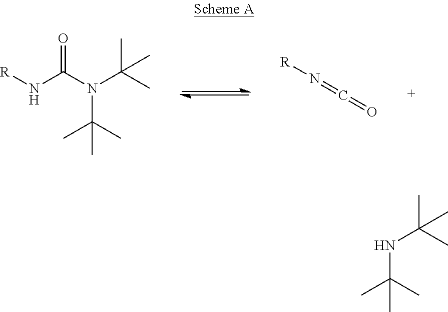

This scheme illustrates that the instable bond between the carbonyl group and the sterically hindered nitrogen atom of the secondary amine is reversibly cleaved under certain conditions, thus forming an isocyanate and the free secondary amine, i.e., the original urea is also reformed, with the equilibrium state strongly depending on the selection of the two substituents on the sterically hindered nitrogen atom.

Sterically hindered urea groups were subsequently used, among other things, for masking isocyanates; see, for example, Hutchby M., Houlden, C. E., Ford, J. G., Tyler, S. N. G., Gagné, M. R., Lloyd-Jones, G. C., Booker-Milburn, K. I., “Hindered ureas as masked isocyanates: facile carbamoylation of nucleophiles under neutral conditions”, Angew. Chem. Int. Ed. 48(46), 8721-8724 (2009)). The first use of this reaction in macromolecules for developing “dynamic” materials is described in Ying, H., Zhang, Y., Cheng, J., “Dynamic urea bond for the design of reversible and self-healing polymers”, Nat. Commun. 5, 3218 (2014)). Here, 2,2,6,6-tetramethylpiperidine carboxylic acid amide and 1-tert-butyl-1-isopropyl-, 1-tert-butyl-1-ethyl-, 1,1-diisopropyl- and 1,1-diethylurea were used for molecular kinetics studies. In addition, a simple polyurea molecule was prepared from 1,3-bis(isocyanatomethyl)cyclohexane and N,N′-di-tert-butylethylenediamine and crosslinked polyurethane-urea polymers were prepared from triethanolamine, hexamethylene diisocyanate, tetraethylene glycol, and four different sterically hindered diamines and examined, showing so-called “self-healing effects” of the crosslinked polymer materials, which were, however, always accompanied by a tensile strength decrease due to the self-healing.

Subsequently, numerous further experiments with corresponding “self-healing” polymers were conducted. For example, Zhang et al. used different ethanolamines, namely 2-tert-butylaminoethanol, 2-isopropyl-aminoethanol und N-butylaminoethanol, for producing so-called “recyclable” poly(urethane-urea) duroplasts (Zhang, Y., Ying, H., Hart, K. R., Wu, Y., Hsu, A. J., Coppola, A. M., Kim, T. A., Yang, K., Sottos, N. R., White, S. R., Cheng, J., “Malleable and recyclable poly(urea-urethane) thermosets bearing hindered urea bonds”, Adv. Mater. 28(35), 7646-7651 (2016)). However, the recycled polymer materials reached the original tensile strength rates at best. Furthermore, Zhang et al. described again the use of ethanolamines as chain extenders for dynamic poly(alkylurea-urethane) networks with improved stress relief (Zhang, L., Rowan, S. J., “Effect of sterics and degree of cross-linking on the mechanical properties of dynamic poly(alkylurea-urethane) networks”, Macromolecules 50(13), 5051-5060 (2017)). Bruce und Lewis examined glass transition temperatures of self-healing poly(urethane-urea)s for the preparation of less soft materials having the same self-healing mechanism (Bruce, A. C., Lewis, C. L., “Influence of glass transition temperature on mechanical and self-healing behavior of polymers bearing hindered urea bonds”, SPE ANTEC, Anaheim, 2017; Anaheim, 2017), and two further articles relate to the use of sterically hindered diamines (in particular N,N′-di-tert-butylethylenediamine) as chain extenders for the preparation of reprocessable poly(urethane-urea) duroplasts with shape memories (Fang, Z., Zheng, N., Zhao, Q., Xie, T., “Healable, reconfigurable, reprocessable thermoset shape memory polymer with highly tunable topological rearrangement kinetics”, ACS Appl. Mater. Interfaces 9(27), 22077-22082 (2017); Wang, Y., Pan, Y., Zheng, Z., Ding, X., “Reconfigurable and reprocessable thermoset shape memory polymer with synergetic triple dynamic covalent bonds”, Macromol. Rapid Commun. 39(10), 1800128 (2018)).

Corresponding disclosures of such “self-healing” polymers can also be found in patent literature, in particular from the above authors as inventors; see, for example, WO 2014/144539 A2, WO 2016/069582 A1, WO 2016/126103 A1, and WO 2016/126756 A1.

However, in aqueous environments, the isocyanate resulting from the reaction shown in Scheme A undergoes a hydrolysis to the corresponding carbamic acid, which is subsequently prone to spontaneous decarboxylation, as is shown in Scheme B below:

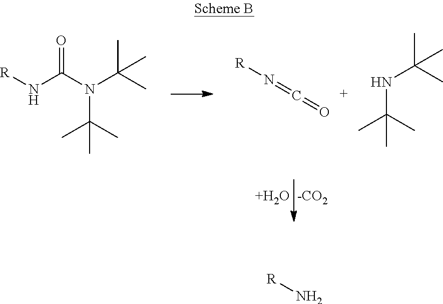

This leads to the two free amines that had initially formed the urea molecule. Such an in situ formation of isocyanates from sterically hindered urea molecules and subsequent hydrolysis of the isocyanates in an aqueous medium allows, among other things, the hydrolysis of polyureas.

For macromolecules, this reaction sequence was first disclosed by Ying et al. (Ying, H., Cheng, J., “Hydrolyzable polyureas bearing hindered urea bonds”, J. Am. Chem. Soc. 136(49), 16974-16977 (2014)), where polyureas and crosslinked poly(urethane-urea) organogels were dissolved in DMF and then hydrolyzed with water, and further works gave evidence for the biocompatibility of hydrogels containing sterically hindered urethane groups for hydrolysis (see Ying, H., Yen, J., Wang, R., Lai, Y., Hsu, J.-L.-A., Hu, Y., Cheng, J., “Degradable and biocompatible hydrogels bearing a hindered urea bond”, Biomater. Sci. 5(12), 2398-2402 (2017)). Later, Cai et al. examined the hydrolysis of a polyurea dissolved in THF with 5% (vol/vol) water and provided evidence for a pH independence of the hydrolysis reaction (Cai, K., Ying, H., Cheng, J., “Dynamic Ureas with fast and pH-independent hydrolytic kinetics”, Chem. Eur. J. 24(29), 7345-7348 (2018); and WO 2017/155958 A1), and recently Chen et al. disclosed the use of polyureas with sterically hindered urea groups for drug release (Chen, M., Feng, X., Xu, W., Wang, Y., Yang, Y., Jiang, Z., Ding, J., “PEGylated polyurea bearing hindered urea bond for drug delivery”, Molecules 24(8), 1538 (2019)), where several PEGylated polyureas dissolved in DMSO were used for creating micelles around an anti-tumor agent (Paclitaxel); the micelles were dissolved in PBS and injected into tumors of experimental animals, where the active agent was released via hydrolysis of the polyurea molecules.

In all these published experiments regarding “self-healing” or “postprocessing” of polyureas and poly(urethane-ureas), of which a great majority only comprises the reversible reaction according to Scheme A and only a few also mention a subsequent hydrolysis reaction, individual mechanical properties were, in rare cases, partially improved, such as the above poly(urethane-ureas) (PUUs) with improved stress relief according to Zhang et al., but mostly modified polymers with worse properties than before or at best the same were obtained. In addition, thermoplastic polyureas, polyurethanes or poly(urethane-urea) polyadducts (TPPUs) have never before been specifically examined under this aspect, and consequently not a single case reported modified polymers with properties that would be better suited for the thermomechanical processing methods mentioned at the beginning.

The still unpublished European application EP 20183957 of one of the applicants describes novel TPUU polyadducts with sterically hindered urea groups of the following formula (I):

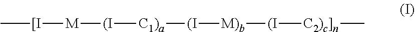

wherein I, M, C₁ and C₂ each represent bivalent radicals that are linked to each other via a urethane or urea moiety, whereof
each I independently represents a bivalent, saturated or unsaturated, aliphatic, alicyclic or aromatic radical with 1 to 20 carbon atoms derived from a diisocyanate;
each M independently represents a bivalent residue of an aliphatic polyether, polyester or polycarbonate derived from a macrodiol having a number average molecular weight Mₙ≥500;
each C₁ independently represents a bivalent, saturated or unsaturated, aliphatic or alicyclic radical with 1 to 30 carbon atoms derived from a diamine or amino alcohol with at least one sterically hindered secondary amino group through removal of one N-linked hydrogen atom each of the diamine or one N-linked and the O-linked hydrogen atoms of the amino alcohol;
each C₂ independently represents a bivalent, saturated or unsaturated, aliphatic, alicyclic or aromatic radical with 1 to 20 carbon atoms derived from a diol, diamine or amino alcohol without sterically hindered amino groups;
wherein, in the radicals I, C₁ and C₂, when more than four carbon atoms are present, optionally at least one of them is replaced by a heteroatom selected from oxygen and nitrogen;
wherein at least one of the radicals I, M, C₁ and C₂ comprises one or more ester moieties; and
a, b and c each independently represent an integer from 0 to 10, and n is a number≥3 representing the number of blocks in the polyadduct;
provided that within each separate block a+c≥1 and in all blocks together at least one a≥1 and at least one c≥1.

For such thermoplastic poly(urethane-urea) (TPUU) polyadducts with sterically hindered urea groups it was found that these macromolecules not only show the reaction shown in Scheme B in a solution or as a hydrogel, as had been known from the state of the art, but also in a solid state on contact with water or in an aqueous environment—even after the material has been processed to solid products, such as by solution casting, foil drawing or the like.

Here, when using the inventive TPUUs in a solid state, the isocyanate formed by opening the unstable urea bond is not completely hydrolyzed to a free amine as in Scheme B, but a part of the isocyanate reacts with a part of the free amine formed by hydrolysis, forming a new, not sterically hindered urea moiety, which results in stable polymer chains with improved thermomechanical properties. Therein, the presence of this reaction—referred to as “recombination reaction”—of a decarboxylated isocyanate with a not yet decarboxylated one is attributed to the lower reaction rate of the hydrolysis reaction of the polymer chains in a solid phase. The following Scheme C shows such a reaction sequence—analogous to Scheme B—for a terminal, sterically hindered urea group:

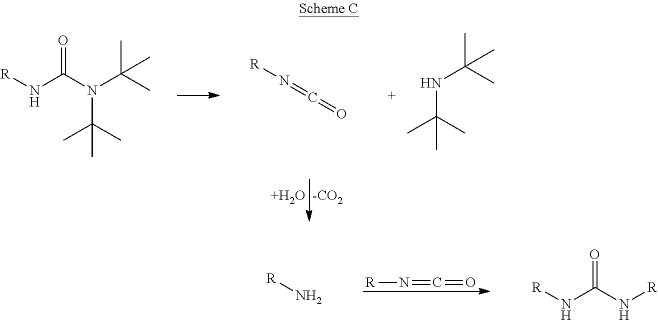

And Scheme D for two urea moieties within a TPUU polymer chain, which moieties are derived from the same secondary diamine sterically hindered on both sides, in the present case from N,N′-di-tert-butylpropylenediamine:

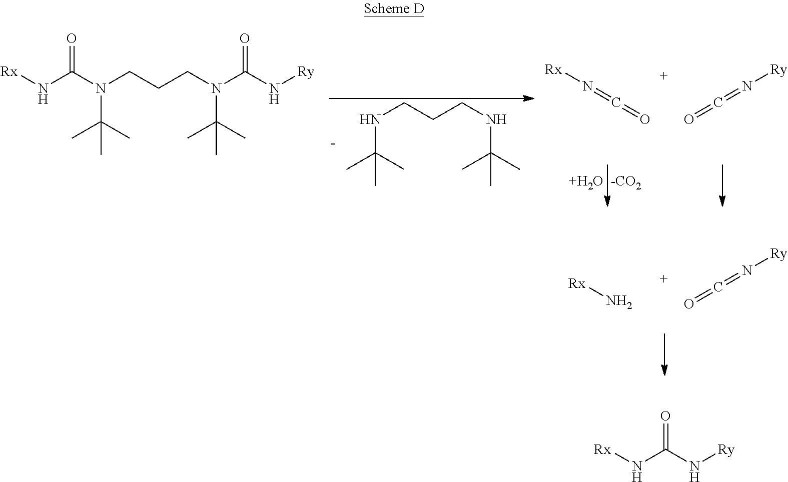

Thereby, on contact of the TPUU with water, a new TPUU polymer is formed, which polymer formally results from the elimination of a diamine sterically hindered on both sides and a carbonyl group from the backbone of the polymer.

The reaction of TPUUs containing radicals of diamines sterically hindered on only one side or amino alcohols is shown in the overleaf Scheme E, wherein X represents O (derived from an amino alcohol) or NH (derived from a diamine):

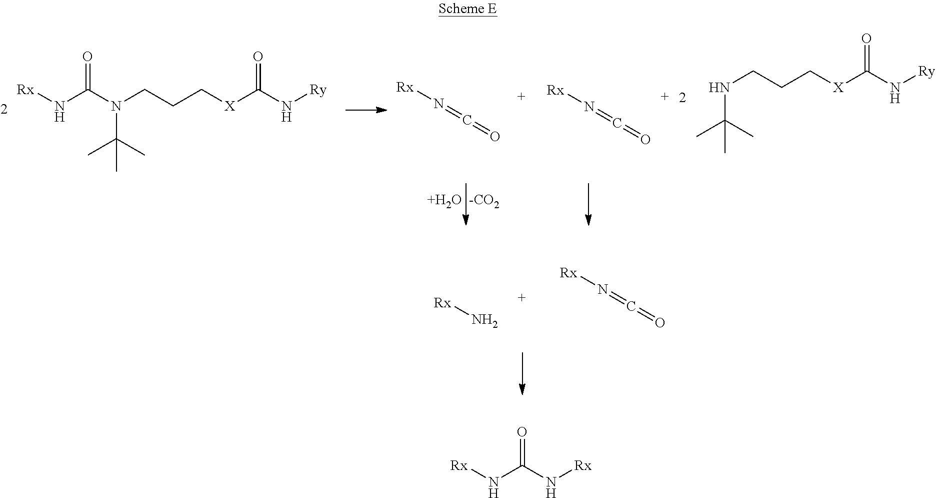

By opening the bonds of one sterically hindered urea moiety each of two TPUU polymer chains, two polymeric isocyanates Rx-N═C═O are formed, one of which undergoes hydrolysis and decarboxylation to a free amine, Rx-NH₂, in the presence of water, subsequently, however, adds to the second isocyanate that is not decarboxylated yet and thus forms a new, stable TPUU polymer with double the chain length of the radical Rx-NH—. In addition, two new TPUU polymers with terminal, sterically hindered secondary amino groups shortened by the length of Rx compared to the starting molecule are formed, which polymers optionally undergo the “self-healing” back reaction with one of the isocyanates to the instable starting polymer as known before, but are not able to form stable urea bonds with isocyanates.

In this way, in an aqueous environment, new polymers are formed from the solid-state TPUUs of Formula (I), which polymers may have longer chain lengths (Scheme C), substantially the same chain lengths (Scheme D) or longer as well as shorter chain lengths (Scheme E) than the starting molecules, depending on the position of the opening instable bond(s) within the backbone, however, in all cases comprise only stable urea bonds that are not sterically hindered after reaction with water.

Only shorter molecules are formed from aqueous solutions of the polyureas mentioned before because the hydrolysis and subsequent decarboxylation of the isocyanates formed by opening instable, sterically hindered urea bonds is much faster in a solution so that they are not stable long enough to undergo a reaction with a free amine. The reaction of solid-state TPUUs on contact with water shown in Scheme D, which results in a free secondary diamine sterically hindered on both sides and a “recombinated” polyurea chain shortened by the length of the diamine and a carbonyl group, would, in a solution, result in only two polymeric free amines in addition to the released sterically hindered diamine, which, of course, cannot undergo any reaction, as is shown in the following Scheme F:

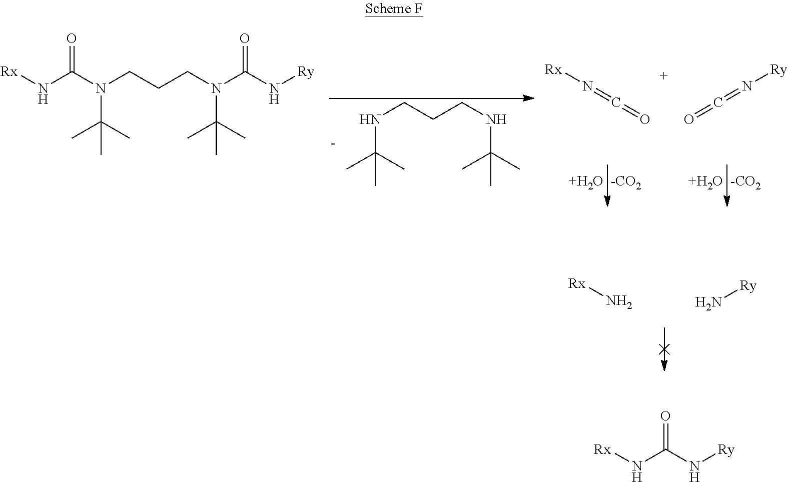

This leads to the formation of two new polymers, the molecular weights of which are, depending on the position of the radical derived from the sterically hindered secondary diamine within the original polymer chain, lower than those of the starting polymer, sometimes considerably lower, up to approximately 50% reduction. This is one of the reasons why almost all older relevant prior art documents on the behavior of “self-healing” polyureas in aqueous solutions report equal thermomechanical properties of the resulting shorter polymers at best, but usually deteriorated ones.

In addition, however, the new polymers, Rx-NH—CO—NH-Ry, resulting according to Scheme D from solid-state TPUUs of Formula (I) on contact with water (or an aqueous environment) have—assuming that only one sterically hindered diamine was contained in the polymer chain—practically have the same molecular weight as before treatment with water and also contain a new urea moiety, the two hydrogen atoms of which are not shielded by bulky radicals and are therefore able to form hydrogen bridges to urea or urethane moieties of a further, adjacent polymer chain. These hydrogen bonds are regarded as the main reason for the improvement of the thermomechanical properties because this effect occurs even when a plurality of sterically hindered urea moieties is contained within the chains of TPUUs of Formula (I) so that “recombination reactions” always form new chains with (sometimes considerable) lower molecular weights.

When processed to solid products, for example, TPUUs of Formula (I) thus show better extensibility and tensile strength values as well as higher melting points after storing the products in water for only a few hours compared to immediately after processing. At the same time, solubility was higher before water treatment, which results in easier processibility of the starting polymers.

Due to the presence of ester moieties in the chains of these TPUUs, the starting polymers as well as the reaction products formed by “recombination” in a suitable aqueous environment, e.g., under physiological conditions, are all cleavable, which offers the advantage of biological degradability.

The components contained in Formula (I) are represented by abbreviations, as is common in polyurethane and polyurea chemistry, with “I” for isocyanate, “M” for macrodiol and “C₁” and “C₂” for two different types of chain extenders which contain amino or OH groups and serve as hard segments for linking isocyanate and macrodiol building blocks via corresponding urethane and/or urea bonds. Of these two chain extenders, C₁, being a diamine sterically hindered on one or both sides or an amino alcohol with a sterically hindered secondary amino group, as defined above, serves for forming the instable urea bond cleavable in an aqueous environment. And C₂ serves, on the one hand, as further hard segment for additionally linking macrodiol building blocks and thus for controlling the chain length between the sterically hindered urea groups formed by C₁, sometimes additionally also for promoting biological degradability, when diamines, diols or amino alcohols, respectively, which contain an ester moiety cleavable under physiological conditions, are selected as monomeric building blocks for introducing C₂ into the TPUU chain. This is particularly advantageous when a polyether without cleavable carboxylate or carbonate ester moieties is selected as macrodiol.

Against this background, it was the object of the present invention to develop a new method for manufacturing vascular prostheses with improved biomechanical recombined properties compared to those of the previously mentioned TPUs.

### DISCLOSURE OF THE INVENTION

In a first aspect, the present invention achieves this object by providing the use of a thermoplastic poly(urethane-urea) polyadduct with sterically hindered urea groups of Formula (I) in an electrospinning method for producing vascular prostheses:

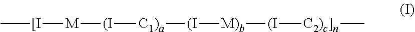

wherein, as described above,
I, M, C₁ and C₂ each represent bivalent radicals that are linked to each other via a urethane or urea moiety, whereof
each I independently represents a bivalent, saturated or unsaturated, aliphatic, alicyclic or aromatic radical with 1 to 20 carbon atoms derived from a diisocyanate;
each M independently represents a bivalent radical of an aliphatic polyether, polyester or polycarbonate derived from a macrodiol having a number average molecular weight Mₙ≥500;
each C₁ independently represents a bivalent, saturated or unsaturated, aliphatic or alicyclic radical with 1 to 30 carbon atoms derived from a diamine or amino alcohol with at least one sterically hindered secondary amino group by removing one N-linked hydrogen atom each of the diamine or one N-linked and the O-linked hydrogen atoms of the amino alcohol;
each C₂ independently represents a bivalent, saturated or unsaturated, aliphatic, alicyclic or aromatic radical with 1 to 20 carbon atoms derived from a diol, diamine or amino alcohol without sterically hindered secondary amino groups;
wherein, in the radicals I, C₁ and C₂, when more than four carbon atoms are present, optionally at least one of them is replaced by a heteroatom selected from oxygen and nitrogen;
wherein optionally at least one of the radicals I, M, C₁ and C₂ comprises one or more ester moieties; and
a, b and c each independently represent an integer from 0 to 10, and n is a number≥3 representing the number of blocks in the polyadduct;
provided that within each separate block a+c≥1 and in all blocks together at least one a≥1 and at least one c≥1.

These thermoplastic poly(urethane-urea) polyadducts of Formula (I) with sterically hindered urea groups represent a new generation of biomaterials, since they have “self-enhancing” properties due to the above-mentioned formation of new polymer chains according to Schemes C to E on contact with water or in an aqueous environment, e.g., on contact with blood. Using such a material, it is possible according to the present invention to produce vascular prostheses by electrospinning, the biomechanical properties of which are superior to those of implants made of the known materials cited at the beginning.

Analogous to EP 20183957, in preferred embodiments of the present invention, at least a part of the ester moieties in the TPUU of Formula (I) is cleavable under physiological conditions, the radicals I, M, C₁ and C₂ as well as any cleavage products thereof are biocompatible and physiologically acceptable, and a temporary vascular prosthesis is prepared by the electrospinning process, which is gradually biodegraded in the body of the recipient and replaced by an endogenous vessel.

In further preferred embodiments of the inventive use, the following applies to TPUUs of Formula (I):
a and c are each independently ≤5 or ≤3; and/or
a and c are each independently ≥1; and/or
b≥1; and/or
b=c or b=a or b+1=a+c; and/or
n≥5 or n≥10 or n≥50.

Here, relatively low values of a and c result in a comparatively high proportion of units M derived from macrodiols, i.e., soft segments, in the TPUUs, which provides for low melting points of the polymers and high flexibility, even at relatively low temperatures, as well as a not overly large number of instable urea bonds in the entire polymer in order not to obtain reaction products with extremely short chains after reaction of the vascular prostheses in the body of the patient and to not impair the thermomechanical properties of the vascular prosthesis.

If a and c are both ≥1, each block contains at least one sterically hindered urea group formed by C₁ as well as a further chain extender C₂, which preferably comprises a cleavable ester moiety, which increases biodegradability.

In TPUUs of Formula (I) with b>1, which is preferably true for the case that a and c are each independently >1, the two chain extender units, C₁ und C₂, are separated by at least one macrodiol unit. This improves controllability of the distance between these units and allows, in particularly preferred embodiments, the provision of a relatively large distance between sterically hindered urea moieties in C₁ and cleavable ester moieties in C₂, so that the recombination reactions of the free amines with non-decarboxylated isocyanate moieties occurring after implantation of the prosthesis do not lead to a premature cleavage of ester groups due to attacks by the free amines.

Embodiments in which b=c or b=a or b+1=a+c mainly offer advantages during the preparation of the TPUUs of Formula (I). For example, in the first two cases, an oligomer or prepolymer with alternating chain extender units C₁ or C₂ and macrodiol units M linked via diisocyanates can be prepared by mixing the corresponding sterically hindered diamine or amino alcohol (for C₁) or the not sterically hindered diamine or amino alcohol or diol (for C₂) with an equimolar amount of a macrodiol with a small molar excess of diisocyanate before the reaction product is reacted with the respectively desired molar amounts of the other chain extender and of diisocyanate. In the case of b+1=a+c, on the one hand, two such oligomers or prepolymers, each containing one chain extender alternating with macrodiol units, can easily be prepared separately and then linked to each other via a diisocyanate. On the other hand, in particularly preferred embodiments in which a, b and c are each 1, equimolar amounts of the monomeric building blocks providing the two chain extenders C₁ and C₂ can be reacted with the double amount of macrodiol and the quadruple amount of diisocyanate (or preferably a small excess of diisocyanate), i.e., in a ratio C₁:C₂:M:I of 1:1:2:4 (or preferably >4, e.g., 4.02 or 4.03), which simplifies synthesis.

For a skilled person, this option with regard to the polymerization reaction sequence means that the order of components within a block, in particular of the components C₁ and C₂, is not limited to that explicitly shown in Formula (I). This means that with values of a and c of >1 each, even if a=c, the two building blocks containing one chain extender each, (I-C₁) and (I-C₂), do not have to alternate within a block in all cases but can also be distributed statistically.

The number of blocks n in the TPUUs of Formula (I) and thus their number-average molecular weights are not particularly limited and can be freely chosen depending on the desired thermomechanical or other physical properties. As well known to the skilled person, the chain length of polyadducts mainly depends on the stoichiometry of the monomeric and prepolymeric building blocks during the polyaddition reactions. Preferably, the number of blocks n is ≥5, more preferably ≥10, and in particular ≥20, ≥50, or ≥100, which improves processibility via electrospinning.

In further embodiments of the invention of the TPUUS of Formula (I):
the radicals I are preferably independently derived from a diisocyanate selected from the group consisting of the following: 1,6-hexamethylene diisocyanate, 4,4′-diisocyanatodicyclohexylmethane, isophorone diisocyanate, 1,3-bis(isocyanatomethyl)cyclohexane, diphenylmethane-4,4′-diisocyanate, L-lysine ethyl ester diisocyanate, even though the diisocyanate are not particularly limited as long as the units I resulting therefrom each have 1 to 30 carbon atoms; and/or
the radicals M are preferably independently derived from a polyether, polyester or polycarbonate from the group consisting of the following: polytetrahydrofuran, polyethylene glycol, polypropylene glycol, polycaprolacton, polylactide, polyglycolide, poly(lactide-co-glycolide), polyhexamethylene carbonate,
   even though the macrodiols are, apart from a number-average molecular weight Mₙ≥ 500 of the units M for providing the polyadducts with suitable molecular weights and thermoplasticity of TPUUs of Formula (I), not particularly limited.

The diamines or aminoalcohols used for introducing the bivalent radicals with at least one sterically hindered secondary amino group each are not particularly limited, either, as long as they have only saturated or unsaturated, aliphatic or alicyclic radicals with 1 to 30 carbon atoms in total in addition to the two nitrogen atoms or the nitrogen atom and the oxygen atom. Herein, this includes all carbon atoms of all radicals that are linked to the nitrogen atom(s) of the aminoalcohol or diamine with (at least) one sterically hindered secondary amino group each, i.e., of the bivalent radical R₁ linking the two amino groups or the amino and hydroxyl groups as well as of the substituent R₂ on the nitrogen atom of the secondary amino group(s), as shown in the following Formulas (II) (diamine) and (III) (aminoalcohol):

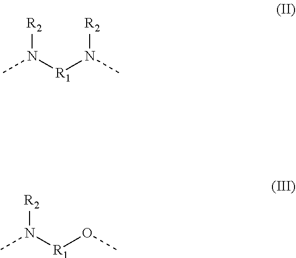

wherein dashed lines each show the linkage to the carbonyl group of a urethane or urea moiety linking the radicals I, M, C₁ and C₂, and at least one of the radicals R₁ and R₂ is a bulky radical causing sterical hindrance of the urea moiety, to which the respective nitrogen atom belongs, within the TPUU. In the case of diamines with only one secondary amino group, one of the radicals R₂ in Formula (II) is hydrogen, and in the case of diamines with two sterically hindered secondary amino groups, either at least radical R₁ in Formula (I) (and optionally also one or both of the radicals R₂) is a bulky radical, or both radicals R₂ (and optionally also radical R₁) are bulky radicals.

In preferred embodiments, the radicals C₁ in the TPUUs of Formula (I) are independently derived from a diamine and selected from radicals of Formula (II) below:

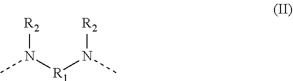

wherein R₁ is selected from bivalent, saturated or unsaturated, aliphatic or alicyclic radicals with 1 to 20 carbon atoms; and
the R₂ are each independently selected from hydrogen and monovalent, bulky, saturated or unsaturated, aliphatic or alicyclic radicals with 1 to 10 carbon atoms, provided that not both R₂ are hydrogen at the same time;
   more preferably from radicals of the above Formula (II), wherein
R₁ is selected from bivalent, saturated or unsaturated, aliphatic or alicyclic radicals with 1 to 10 carbon atoms; and/or
each R₂ is independently selected form monovalent, bulky, saturated or unsaturated, aliphatic or alicyclic radicals with 1 to 10 carbon atoms;
   particularly preferred from radicals of Formula (II), wherein
R₁ is selected form C₁-C₁₀-alkylene and C₄-C₁₀-cycloalkylene radicals; and/or
each R₂ is independently selected form 1,1-dimethyl-substituted, saturated or unsaturated C₁-C₆-alkyl radicals and 1-methyl-substituted C₃-C₆-cycloalkyl radicals; and in particular form radicals of Formula (II), wherein
R₁ is selected from C₂-C₆-alkylene and C₅-C₆-cycloalkylene radicals; and/or
each R₂ is independently selected from isopropyl, tert-butyl, 1,1-dimethylpropyl and 1-methylcyclohexyl.

These preferred selection options from these physiologically acceptable radicals of secondary diamines sterically hindered on both sides and having relative short chains result in having short-chain hard segments C₁ in the TPUUs of Formula (I) compared to the soft segments M with higher molecular weights, which promotes their thermoplasticity and subsequently also the probability of the “recombination reaction” according to the above Scheme D during contact of the vascular prosthesis electrospun therefrom with water.

Furthermore, in preferred embodiments, at least one of the radicals C₂ of the TPUUs comprises one or more ester moieties, which are particularly preferably each independently derived from a diol selected from the following group: bis(hydroxyethyl) terephthalate, bis(hydroxypropyl) carbonate, 2-hydroxyethyl lactate, and 2-hydroxyethyl glycolate, because they have relatively short chains and are physiologically acceptable.

Particularly preferably, in the TPUUs of Formula (K) b+1=a+c so that the polyadduct corresponds to the following formula (IV):

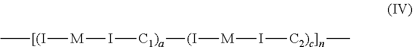

wherein
a and c are each independently selected from 1 to 3, or
a and c are each 1; and
n≥5 or n≥10 or n≥20.

Thus, the polyaddition method for producing the TPUUs is particularly easily controllable, wherein preferably the desired macrodiol (or various different ones) is first reacted in a solution in a suitable anhydrous solvent with a bit more than the double amount of diisocyanate in order to obtain prepolymers, or intermediates, terminated with isocyanate on both sides, with the chain structure I-M-I, whereafter the two reactants introducing the chain extenders C₁ and C₂ are successively added in molar amounts, the sum of which corresponds to the molar amount of the macrodiol.

Due to the lower reactivity of sterically hindered amines and the instability of the urea compounds formed by them, in particular in solutions, the chain extender moiety C₁ is preferably introduced into the TPUUs as last building block in the polyaddition process. Thus, the isocyanate-terminated prepolymer with the chain structure I-M-I is preferably first reacted with the diol, diamine or amino alcohol without sterically hindered secondary amino groups containing C₂, which, for example, results in isocyanate-terminated intermediates with the chain structure I-M-I-C₂—I-M-I in case of a reaction of half the molar amount of chain extender with reference to the macrodiol units M. These are subsequently reacted with the chain extender reagent containing C₁, i.e., the diamine or amino alcohol containing sterically hindered amino groups, which, in case of equimolar amounts of the two chain extenders, results in a TPUU polymer with blocks of the structure of Formula (V):

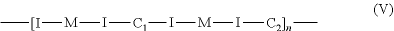

i.e., TPUUs of Formula (I), wherein a=b=c=1. Here, the value of n, i.e., the number of blocks, and thus the chain length and the molecular weight of the TPUUs depend, in addition to the purity of the monomers as mentioned before, mainly on their stoichiometry as well as on the reaction sequence and here mainly on the order in which the various monomer or prepolymer components are added. Preferably is n≥5, more preferably ≥10, more preferably ≥20.

In further preferred embodiments, the present invention is characterized in that in the electrospinning process, a solution of the TPUU in an organic solvent or solvent mixture, particularly preferably a solution in hexafluoroisopropanol, in an electrospinning device that comprises a high-voltage generator, a syringe pump, a syringe with a blunt end as an electrode, a grounded, electrically conductive rotating steel mandrel as a collector electrode, and optionally an auxiliary electrode, is injected by means of the syringe into the electric field built up between the electrodes, and the polymer fibers that are formed as continuous nanofibers are wound onto the rotating mandrel as a tube suitable as a vascular prosthesis. Such a procedure has already proven itself in the past for the preparation of vascular prostheses.

In particularly preferred embodiments, a solution of a mixture of the TPUU of Formula (I) and at least one further polymer is used and tubes consisting of the mixture are prepared during electrospinning. It has been found in the experiments of the inventors that during the preparation of prostheses which consist of the TPUU of Formula (I) alone, the formation of fibers works less well and the porosity of the material is lower. For the latter reason, the accessibility of the wound fibers for aqueous media such as blood is limited, which reduces the biodegradability of the prosthesis. The at least one further polymer is preferably used in a proportion of at least 10% by weight, at least 15% by weight, at least 20% by weight, at least 25% by weight, at least 30% by weight or optionally also at least 50% by weight of the mixture in order to improve the above properties of the mixture.

Furthermore, according to the present invention, a mixture of the TPUU of Formula (I) and a TPU is preferably used, more preferably a biodegradable TPU, which has already proven to be suitable for the preparation of vascular prostheses in the past, wherein according to the present invention, in particular a polyether urethane, such as a polyadduct of polytetrahydrofuran, bis(hydroxyethyl) terephthalate and hexamethylene diisocyanate, is used as TPU in the mixture with the TPUU of Formula (I), e.g., a mixture of 50% by weight each of TPUU and TPU.

In a second aspect, the present invention also provides the vascular prostheses obtainable by the inventive electrospinning process of the first aspect using TPUUs of Formula (I), which are characterized by good processability and biodegradability, as will be shown by the examples below.

### SHORT DESCRIPTION OF THE DRAWINGS

Below, the present invention will be described in more detail by means of illustrative, non-limiting exemplary embodiments and with reference to the accompanying drawings, showing the following:

FIG. 1 shows two micrographs (FIGS. 1a and 1b) of the prosthesis from Example 1A of the present invention.

FIG. 2 shows a photograph (FIG. 2a) and two micrographs (FIGS. 2b and 2c) of the prosthesis from Example 2A of the present invention.

FIGS. 3 to 5 are graphical representations of the results of tensile strength tests on the prostheses of Comparative Example 1 and Examples 1 to 5.

FIGS. 6 to 8 are graphical representations of the results of biodegradability tests of the TPUU contained in the prostheses from Example 1A.

FIGS. 9 and 10 show the results of cytotoxicity tests of the TPUU from example 1A for HUVECs and macrophage cells (FIGS. 8a and 8b) and the TBEDA monomer used for its preparation for HUVECs (FIG. 9).

FIGS. 11a and 11b are micrographs taken during tests of the adhesion and proliferation of EPCs on the prosthesis from Example 1A.

And FIGS. 12A-H and 13I-N are photographs of various stages of in vivo biocompatibility tests of the vascular prosthesis from Example 2A.

### EXAMPLES

As representative examples of particularly preferred embodiments of the present invention, a TPUU, mixtures of the TPUU and a TPU, and the TPU alone were processed by means of electrospinning into tubes made of continuous fibers, which were then tested for their suitability as vascular prostheses. Before that, the polymer was prepared using the preferred reaction procedure outlined above, i.e., by sequential reactions of the individual components, first producing prepolymers isocyanate-terminated on both sides or intermediate products with the chain structure I-M-I, which are successively reacted with the reactants introducing the chain extenders C₂ and then C₁. Here, the TPU from Synthesis Example 14 corresponds to “TPU4” of the thermoplastic polyether urethanes disclosed in Baudis et al. (2012; see above) and was made from polytetrahydrofuran, bis(hydroxyethyl) terephthalate and hexamethylene diisocyanate.

### Synthesis Example 1

By using the standard Schlenk line with argon as the inert gas, first, pre-dried poly(tetrahydrofuran) (pTHF) (Mₙ≈1 kDa, 6.059 g, 6.1 mmol, 1.00 eq., 19 ppm H₂O) as the macrodiol was weighed into a reaction flask and dried at 60° C. under high vacuum for 1 hour. Subsequently, 5 ml abs. dimethylformamide (DMF), followed by hexamethylene diisocyanate (HMDI) (2.111 g, 12.6 mmol, 2.07 eq.) in 5 ml abs. DMF were added to the dried, melted pTHF. After adding 2 drops (about 0.04 ml) of tin(II) 2-ethylhexanoate as a catalyst, the reaction mixture was magnetically stirred at 60° C. under protective argon atmosphere for 3 hours. Then, bis(hydroxyethyl) terephthalate (BHET) (0.770 g, 3.03 mmol, 0.5 eq.) was added as the diol for introducing C₂ as a solution in 5 ml abs. DMF. After further stirring at 60ºC for 3 hours, the reaction mixture was cooled to room temperature, after which N,N′-di-tert-butylethylenediamine (TBEDA) (0.522 g, 3.03 mmol, 0.5 eq.) was added as a secondary diamine for introducing C₁ that was sterically hindered on both sides. After each addition, the transfer vessels or syringes, respectively, were each flushed with 5 ml abs. DMF. The reaction solution was stirred overnight. To recover and purify the prepared TPUU, the reaction mixture was diluted with DMF and added dropwise to ten times the volume of diethyl ether, whereby a colorless precipitate was precipitated which was subsequently dried and characterized by GPC and NMR.

By reacting these reactants at a ratio of C₁:C₂:M:I=1:1:2:4 (or 4.14, respectively), a TPUU of Formula (IV) above was obtained, wherein a=b=c=1, i.e., a TPUU of Formula (V):

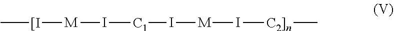

The value n was calculated from the weight average molecular weight (M_{w}), as determined using gel permeation chromatography (GPC), and the molar mass of the blocks. The M_{w} of the obtained TPUU was determined to be about 65.6 kDa, and the molar mass of one block was about 3.1 kDa, resulting in a value for n of about 21.

The precise structure of this TPUU of Formula (V) is depicted overleaf. The average value m of the units M of polyTHF with a number average molecular weight Mₙ of about 1 kDa is thus about 14. Furthermore, the portions corresponding to the units C₁, C₂, M and I are depicted below.

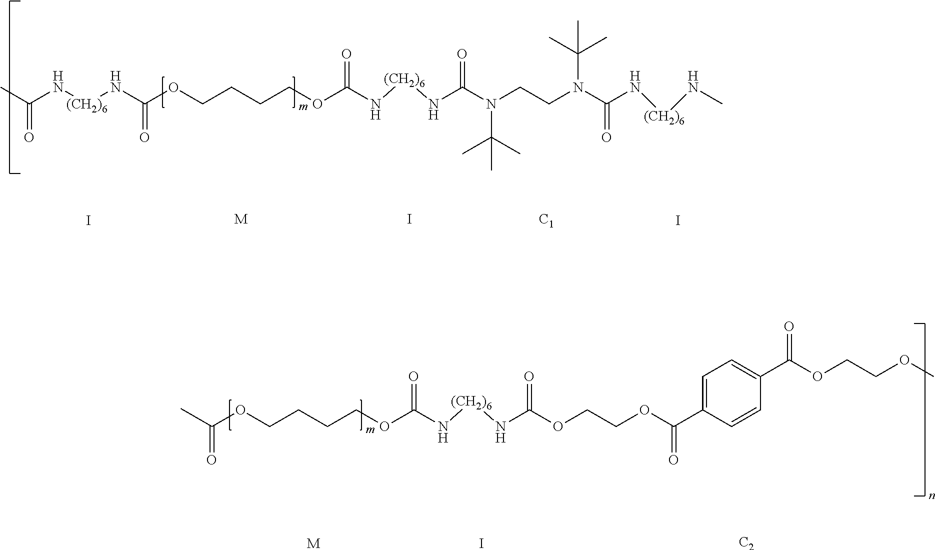

### Synthesis Example 2

To prepare another embodiment of a TPUU of Formula (I), Synthesis Example 1 was substantially repeated, wherein the molar ratio of the chain extender units C₂ and C₁ that were sequentially incorporated into the polymer chains was changed from 1:1 to 3:1. That is, at first, instead of 0.5 equivalents bis(hydroxyethyl) terephthalate 0.75 equivalents were reacted, and then instead of 0.5 equivalents N,N′-di-tert-butylethylenediamine only 0.25 equivalents were reacted.

By reacting the four reactants at a ratio of C₁:C₂:M:I=0.5:1.5:2:4 (or 4.14, respectively), a TPUU of Formula (IV) was obtained:

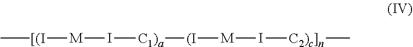

wherein a=1 and c=3. The four portions that each contain one of the two chain extenders are randomly distributed within a block.

Using GPC, the M_{w} of the TPUU thus obtained was determined to be about 62.8 kDa, and the molar mass of a block was about 6.3 kDa, resulting in a value for the number of blocks n of about 10.

### Synthesis Example 3

To prepare another embodiment of a TPUU of Formula (I), Synthesis Example 2 was substantially repeated, wherein in this case, the molar ratio between C₂ and C₁ was reversed. That is, at first, instead of 0.5 equivalents bis(hydroxyethyl) terephthalate only 0.25 equivalents were reacted, and then instead of 0.5 equivalents N,N′-di-tert-butylethylenediamine 0.75 equivalents were reacted.

By reacting the four reactants at a ratio of C₁:C₂:M:I=1.5:0.5:2:4 (or 4.14, respectively) a TPUU of Formula (IV) was obtained:

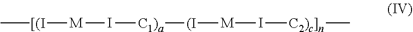

wherein a=3 and c=1, and the four portions containing the chain extenders are randomly distributed within a block.

Using GPC, the M_{w} of the TPUU thus obtained was determined to be about 74.4 kDa, and the molar mass of a block was about 6.1 kDa, resulting in a value for the number of blocks n of about 12.

In the following Synthesis Examples 4 to 13, by reacting the reactants anew at a ratio of C₁:C₂:M:I=1:1:2:4, similarly to the abovementioned Example 1—but by varying the components—other TPUUs of Formula (V) were obtained, wherein a=b=c=1, i.e., TPUUs of Formula (V):

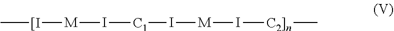

### Synthesis Example 4

Synthesis Example 1 was substantially repeated, wherein instead of poly(tetrahydrofuran) (pTHF) (Mₙ≈1 kDa) as the macrodiol a poly(hexamethylene carbonate)diol (pHMC) having a number average molecular weight Mₙ of about 1.2 kDa in abs. DMF was reacted with HMDI, BHET, and finally TBEDA as the secondary diamine sterically hindered on both sides, wherein by precipitating with diethyl ether the desired polyadduct was obtained as a flaky, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the macrodiol radical derived from pTHF is replaced by the corresponding radical M derived from pHMC of the formula below, wherein the value for m is about 9.

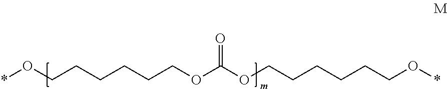

Using GPC, the M_{w} of the TPUU thus obtained was determined to be about 128 kDa, and the molar mass of a block was about 3.6 kDa, resulting in a value for the number of blocks n of about 36.

### Synthesis Example 5

Synthesis Example 1 was substantially repeated, wherein instead of poly(tetrahydrofuran) (pTHF) as the macrodiol a poly(caprolactone) diol, more precisely poly(caprolactone) diol-540 (pCL540) having a number average molecular weight Mₙ of about 540 Da was reacted with HMDI, BHET, and finally TBEDA as the secondary diamine sterically hindered on both sides, wherein by precipitating with diethyl ether the desired polyadduct was obtained as a flaky, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the macrodiol radical derived from pTHF is replaced by the corresponding radical M derived from pCL540 of the formula below, wherein the value for each m≈2.

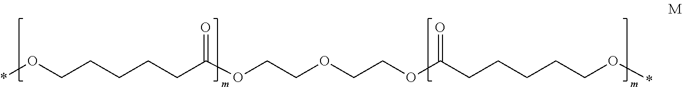

Using GPC, the M_{w} of the TPUU thus obtained was determined to be about 62.4 kDa, and the molar mass of a block was about 2.2 kDa, resulting in a value for the number of blocks n of about 28.

### Synthesis Example 6

Synthesis Example 5 was substantially repeated, wherein instead of poly(tetrahydrofuran) (pTHF) as the macrodiol, again, a poly(caprolactone) diol, but in this case poly(caprolactone) diol-2000 (pCL2000) having a number average molecular weight Mₙ of about 2.2 kDa was reacted with HMDI, BHET, and TBEDA, wherein by precipitating with diethyl ether the desired polyadduct was obtained as a sticky, colorless precipitate that was removed from the reaction vessel with a spatula and dried.

The structure of this TPUU corresponds to that of the TPUU of Synthesis Example 5, but having accordingly higher values for the degree of polymerization m of the radical M derived from pCL2000, namely about 9 each.

Using GPC, the M_{w} of the TPUU thus obtained was determined to be about 56.4 kDa, and the molar mass of a block was about 5.4 kDa, resulting in a value for the number of blocks n of about 10.

### Synthesis Example 7

Synthesis Example 4 was substantially repeated, wherein instead of hexamethylene diisocyanate (HMDI) 4,4′-diisocyanatodicyclohexylmethane (H12MDI) as the diisocyanate was reacted with pHMC, BHET, and finally TBEDA as the secondary diamine sterically hindered on both sides, wherein by precipitating with diethyl ether the desired polyadduct was obtained as a flaky, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein radical M derived from pTHF is replaced by radical M derived from pHMC (m=9) and radical I derived from HMDI is replaced by radical I derived from H12MDI of the following formulae.

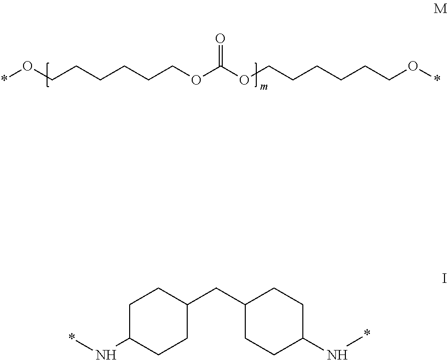

Using GPC, the M_{w} of the TPUU thus obtained was determined to be about 46.2 kDa, and the molar mass of a block was about 3.5 kDa, resulting in a value for the number of blocks n of about 13.

### Synthesis Example 8

Synthesis Example 1 was substantially repeated, wherein instead of bis(hydroxyethyl)terephthalate (BHET) 1,4-butanediol (BDO) as the chain extender for introducing C₂ was reacted with pTHF, HMDI, and TBEDA as the secondary diamine sterically hindered on both sides, wherein by precipitating with diethyl ether the desired polyadduct was obtained as a sticky, colorless precipitate that was removed from the reaction vessel with a spatula and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from BHET is replaced by the radical C₂ derived from BDO of the following formula.

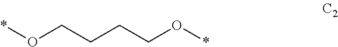 [SMILES: *OCCCCO*]

Using GPC, the M_{w} of the TPUU thus obtained was determined to be about 54.7 kDa, and the molar mass of a block was about 3.0 kDa, resulting in a value for the number of blocks n of about 18.

### Synthesis Example 9

Synthesis Example 1 was substantially repeated, wherein instead of BHET bis(3-hydroxypropyl) carbonate (BHPC) as the chain extender for introducing C₂ was reacted with pTHF, HMDI, and TBEDA as the secondary diamine sterically hindered on both sides, wherein by precipitating with diethyl ether the desired polyadduct was obtained as a sticky, colorless precipitate that was removed from the reaction vessel with a spatula and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from BHET is replaced by the corresponding radical C₂ derived from BHPC of the following formula.

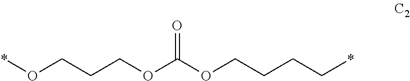 [SMILES: *CCCCOC(=O)OCCCO*]

Using GPC, the M_{w} of the TPUU thus obtained was determined to be about 163 kDa, and the molar mass of a block was about 3.1 kDa, resulting in a value for the number of blocks n of about 53.

### Synthesis Example 10

Synthesis Example 1 was substantially repeated, wherein instead of BHET 2-hydroxyethyl lactate (ethylene glycole lactate, EGLA) as the chain extender for introducing C₂ was reacted with pTHF, HMDI, and TBEDA as the secondary diamine sterically hindered on both sides, wherein by precipitating with diethyl ether the desired polyadduct was obtained as a sticky, colorless precipitate that was removed from the reaction vessel with a spatula and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from BHET is replaced by the corresponding radical C₂ derived from EGLA of the following formula.

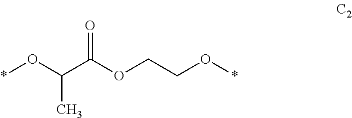 [SMILES: *OCCOC(=O)C(C)O*]

Using GPC, the M_{w} of the TPUU thus obtained was determined to be about 58.9 kDa, and the molar mass of a block was about 3.0 kDa, resulting in a value for the number of blocks n of about 19.

### Synthesis Example 11

Synthesis Example 1 was substantially repeated, wherein instead of N,N′-di-tert-butylethylenediamine (TBEDA)N-tert-butylaminoethanol (TBAE) as the chain extender for introducing C₁, i.e., an amino alcohol with only one sterically hindered secondary amino group, was reacted with pTHF, HMDI, and BHET, wherein by precipitating with diethyl ether the desired polyadduct was obtained as a flaky, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from TBEDA is replaced by the corresponding radical C₁ derived from TBAE of the following formula.

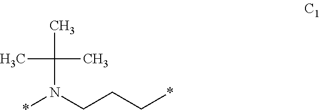 [SMILES: *CCCN(*)C(C)(C)C]

Using GPC, the M_{w} of the TPUU thus obtained was determined to be about 103 kDa, and the molar mass of a block was about 3.1 kDa, resulting in a value for the number of blocks n of about 33.

### Synthesis Example 12

Synthesis Example 1 was substantially repeated, wherein instead of N,N′-di-tert-butylethylenediamine (TBEDA) N,N′-diisopropylethylenediamine (IPEDA) as a chain extender for introducing C₁, i.e., a diamine with a slightly weaker sterically hindered secondary amino group, was reacted with pTHF, HMDI, and BHET, wherein by precipitating with diethyl ether the desired polyadduct was obtained as a flaky colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from TBEDA is replaced by the corresponding radical C₁ derived from IPEDA of the following formula.

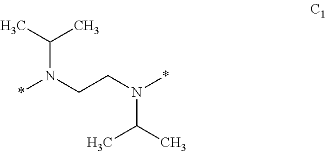 [SMILES: *N(CCN(*)C(C)C)C(C)C]

Using GPC, the M_{w} of the TPUU thus obtained was determined to be about 85.3 kDa, and the molar mass of a block was about 3.1 kDa, resulting in a value for the number of blocks n of about 28.

### Synthesis Example 13

Synthesis Example 1 was substantially repeated, wherein instead of N,N′-di-tert-butylethylenediamine (TBEDA) 2,6-dimethylpiperazine (2,6-DMP) as a chain extender for introducing C₁, i.e., a cyclic diamine with only one sterically hindered secondary amino group (the second amino group is also secondary, but not sterically hindered according to the invention as shown by the later tests for self-enhancing properties for the TPPU from Synthesis Example 16) was reacted with pTHF, HMDI, and BHET, wherein by precipitating with diethyl ether the desired polyadduct was obtained as a solid, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from TBEDA is replaced by the corresponding radical C₁ derived from 2,6-DMP of the following formula.

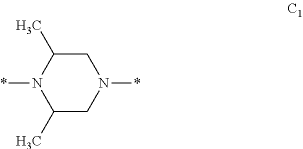 [SMILES: *N1CC(C)N(*)C(C)C1]

Using GPC, the M_{w} of the TPUU thus obtained was determined to be about 153.4 kDa, and the molar mass of a block was about 3.1 kDa, resulting in a value for the number of blocks n of about 49.

### Synthesis Example 14

Similarly to Synthesis Example 1, a TPU was prepared by reacting pTHF, (Mₙ≈1 kDa), HMDI, and BHET, wherein no sterically hindered secondary diamine for introducing C₁ was added, but an amount of BHET equimolar to the amount of pTHF was used. As a result, the ratio of radicals in the polyadduct was C₂:M:I=1:1:2, which is thus a thermoplastic poly(urethane) (TPU; without urea moieties) of the following Formula (VI):

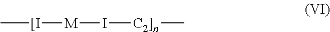

Using GPC, the M_{w} of the TPUU thus obtained was determined to be 46 kDa, and the molar mass of a block was about 1.6 kDa, resulting in a value n of about 29.

Due to the cleavable ester bonds in C₂, this TPU is degradable under physiological conditions, but, does not have any self-enhancing properties.

### Synthesis Example 15

Synthesis Example 1 was substantially repeated, wherein instead of N,N′-di-tert-butylethylenediamine (TBEDA), piperazine (Pip) as the chain extender for introducing C₁, i.e., a cyclic diamine with two secondary amino groups, but not sterically hindered according to the invention, as shown by the later tests for self-enhancing properties, was reacted with pTHF, HMDI, and BHET, wherein by precipitating with diethyl ether the desired polyadduct was obtained as a solid, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from TBEDA is replaced by the corresponding radical C₁ derived from Pip of the following formula.

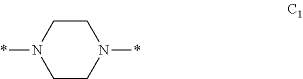 [SMILES: *N1CCN(*)CC1]

Using GPC, the M_{w} of the TPUU thus obtained was determined to be about 325.5 kDa, and the molar mass of a block was about 3.1 kDa, resulting in a value for the number of blocks n of about 105.

### Synthesis Example 16

Synthesis Example 1 was substantially repeated, wherein instead of N,N′-di-tert-butylethylenediamine (TBEDA), 2,5-dimethylpiperazine (2,5-DMP) as the chain extender for introducing C₁, i.e., again a cyclic diamine with two secondary amino groups, neither of which sterically hindered according to the invention, as shown by the later tests for self-enhancing properties, was reacted with pTHF, HMDI, and BHET, wherein by precipitating with diethyl ether the desired polyadduct was obtained as a solid, colorless precipitate that was removed by filtration and dried.

Consequently, the structure of this TPUU corresponds to the depicted structure for the TPUU of Synthesis Example 1 of Formula (V) —[I-M-I-C₁—I-M-I-C₂]ₙ— above, wherein the radical derived from TBEDA is replaced by the corresponding radical C₁ derived from 2,5-DMP of the following formula.

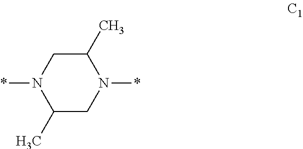 [SMILES: *N1CC(C)N(*)CC1C]

Using GPC, the M_{w} of the TPUU thus obtained was determined to be about 51.8 kDa, and the molar mass of a block was about 3.1 kDa, resulting in a value for the number of blocks n of about 17.

### Examples—Preparation of Vascular Prostheses by Electrospinning

### Example 1A—TPPU

The TPUU prepared in Synthesis Example 1 was dissolved in hexafluoroisopropanol and, using an electrospinning apparatus comprising a high-voltage generator, a syringe pump, a syringe with a blunt-ended 21 G needle as an outlet nozzle, a grounded electrically conductive rotatable steel mandrel as a collector electrode or a Teflon mandrel as a collector with an auxiliary electrode, a grounded, electrically conductive rotating steel mandrel as a collector electrode or a Teflon mandrel as a collector with an auxiliary electrode, processed into a continuous nanofiber that was wound into a tube on the rotating mandrel. The distribution of potentials between the individual electrodes were individually adjustable, and the potentials of the rotating mandrel and the auxiliary electrode were set to achieve an optimum fiber deposition rate. The distance between the needle tip and the mandrel was 8 cm. The electrospinning device was placed as a whole in a Faraday cage and operated in a class 1000 clean room at a temperature of 24° C. and a RH of 34%, with the inflow of the polymer solution into the syringe being set to 0.7 ml/h at the syringe pump and a voltage of 12 kV being applied to the polymer exiting the needle tip.

### Example 2A—TPUU and TPU 50:50

A solution of a mixture of 50% by weight each of the TPUU from Synthesis Example 1 and the TPU from Synthesis Example 14 in hexafluoroisopropanol was processed into electrospun tubes in a similar manner as in Example 1A, however, with a voltage of 8.5 kV being applied to the polymer mixture emerging from the needle tip.

The electrospun tubes thus obtained on the mandrel of both examples had an inner diameter of 1.5 mm and a wall thickness of about 300 μm and were dried in a vacuum at 40° C. for 2 h each to remove residual solvent.

### Examples 1B, 2B and 3 to 5, Comparative Example 1

According to initial tensile strength tests with the two electrospun tubes from Example 1 and Example 2, a series of tubes was tested with hexafluoroisopropanol solutions of the TPUU from Synthesis Example 1, of the TPU from Synthesis Example 14 and of mixtures of the two in various mixing ratios in a manner analogous to Example 2A. The mixing ratios were as follows.
Example 1B: 100% by weight TPUU
Example 2B: 50% by weight TPUU, 50% by weight TPU
Example 3: 30% by weight TPUU, 70% by weight TPU
Example 4: 10% by weight TPUU, 90% by weight TPU
Example 5: 5% by weight TPUU, 95% by weight TPU
Comparative Example 1: 100% by weight TPU

### Testing the Physical and Biomechanical Properties

The dry tubes obtained in Examples 1 to 5 and Comparative Example 1 were each cut into rings with a width of 2 mm and tested for their properties in the following way.

### Fiber Formation, Porosity

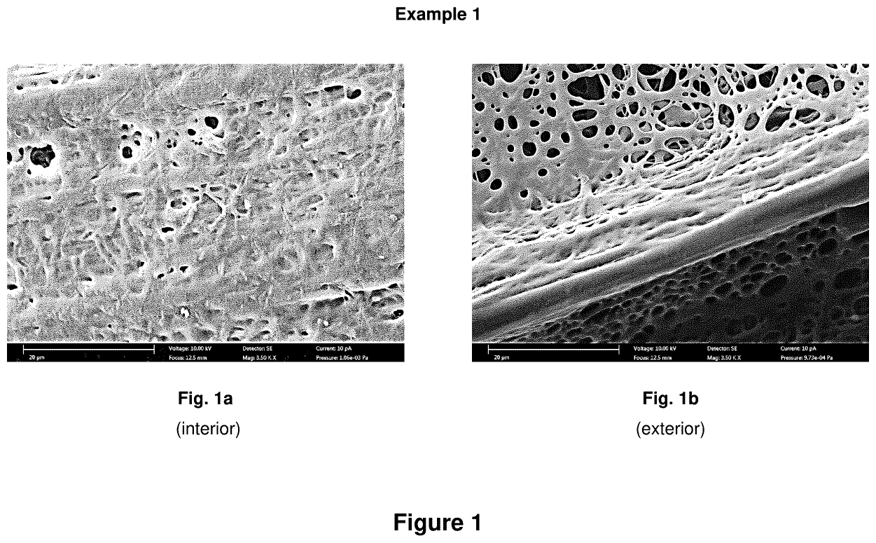

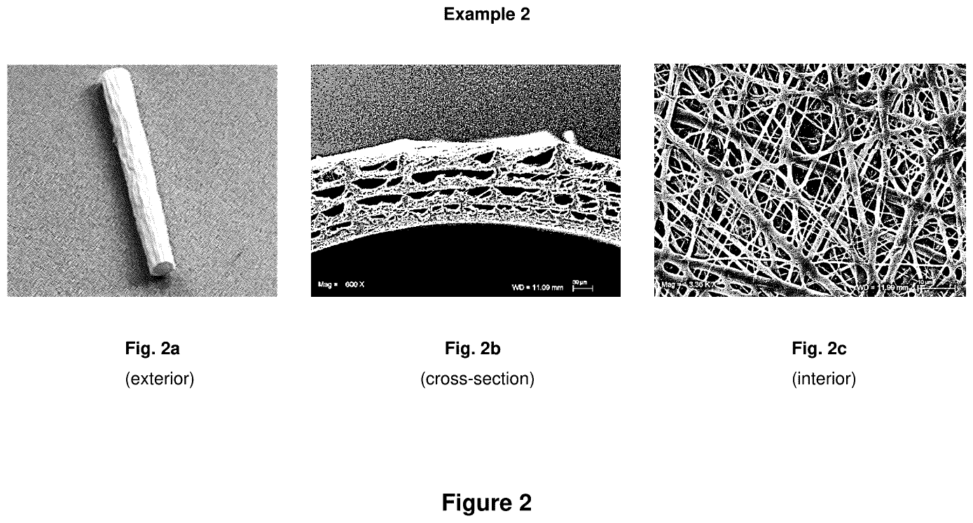

For a surface characterization of the prostheses from Example 1A and Example 2A, the inner and outer surfaces were each coated with gold-palladium. The luminal morphology was determined using an EVO 10 scanning electron microscope from Zeiss, Germany, at an accelerating voltage of 10 kV at 3500× magnification. The fiber structure on the inner and outer surfaces of the vascular prosthesis from Example 1A are shown in FIGS. 1a and 1b, respectively, and FIG. 2 shows views of the fiber structure at the outer (FIG. 2a), the cross-sectional (FIG. 2b), and the inner surface (FIG. 2c) of the prosthesis from Example 2A.

In FIGS. 1a and 1b it can be seen that although tubes suitable as vascular prostheses could certainly be electrospun in Example 1A using the TPUU from Synthesis Example 1 alone, there was no optimal fiber formation during electrospinning, as evidenced by the relatively low porosity of the spun tubes. By contrast, electrospinning of the 50:50 mixture of the TPUU and a conventional TPU in Example 2A resulted in the prosthesis shown in FIG. 2a of a fibrous web with well-defined individual fibers and well-defined voids between them, so that it has significantly improved porosity compared to that from Example 1A. The latter increases, after subsequent implantation of the prostheses, the accessibility of the individual fibers for blood and thus improves their biodegradability over time.

### Tensile Strength

A) Prostheses from Example 1A

Of the rings with a width of 2 mm from the prostheses obtained in Example 1A, fifteen were stored dry or in a physiological saline for up to 34 d at room temperature.

After 1, 2, 7, 14, and 34 d of storage, tensile tests were performed on three wet-stored rings three dry-stored rings by elongating them in the circumferential direction at a speed of 10 mm/min up to the maximum travel of the machine (12 mm) using an ElectroForce® TestBench by Bose. The tests were performed in a Premiere Tissue Floating Bath XH-I 003 at 37° C., and force-elongation curves were plotted using WinTest software and compared to a Matlab-based analysis table.

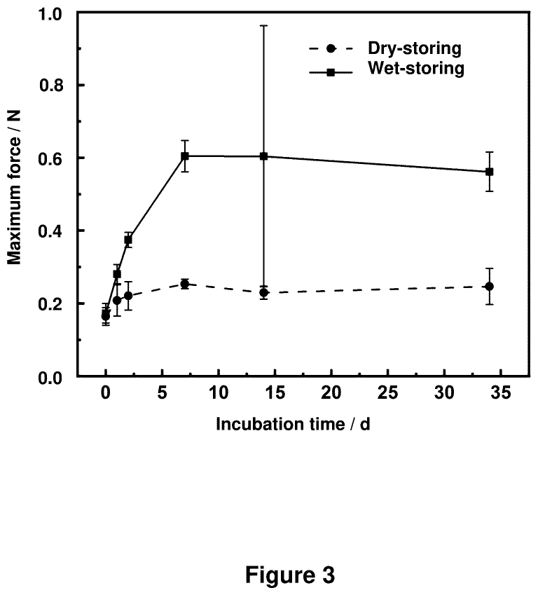

FIG. 3 shows a comparison of the obtained results for the dry-stored and the water-treated rings. It can be seen that after only 1 d, the tensile strength stated as maximum usable force (in N) of the water-treated rings is, on average, approximately one third higher than that of the dry-stored. This effect is further increased during storing, until after 7 d the water-treated rings have an almost three times higher tear strength than the dry-stored. Until then, all rings broke before reaching the, in the present case, maximum travel of 12 mm, but the water-treated rings did not break anymore after day 7. Later tests after 14 d or 34 d gave substantially the same results as after 7 d.

B) Prostheses from Example 2A

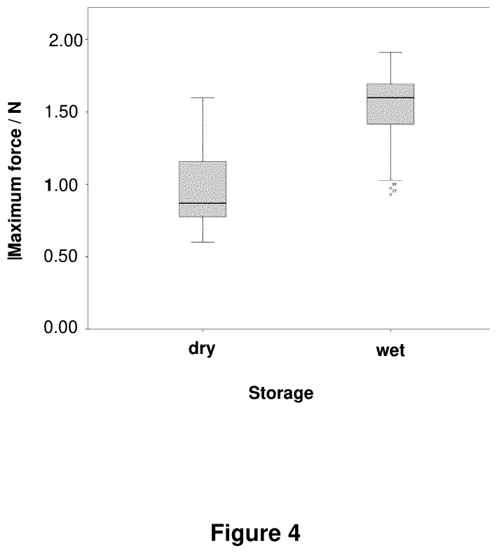

Based on the results obtained with the above prostheses from Example 1A, the rings of the prostheses from Example 3A were each stored dry or in a physiological saline for 7 d at room temperature. Subsequently, the maximum tensile strength until the rings broke was measured as above using an ElectroForce® TestBench by Bose. The results are graphically shown in FIG. 4 as means plus standard deviation.

The graph shows that the dry-stored rings have, on average, a maximum force of 0.98 N, but the wet-stored ones withstood 1.55 N. Thus, the tensile strength of the polymer mixture of TPUU and TPU was also improved by approximately half with storing for 7 d, which proves the self-enhancing effect of the TPUU of Formula (I) at contact with an aqueous environment.

C) Protheses from Examples 1B, 2B and 3 to 5 and Comparative Example 1

Due to the fact that by adding 50% by weight of TPU from Synthesis Example 14 without any self-enhancing effect to the TPUU of Formula (I) from Synthesis Example 1, the tensile strength of the rings (expressed as maximum force) from Example 2A was higher than that of the rings from Example 1A of only TPUU, it was to be assumed that this is to be attributed to different framework conditions during electrospinning. Therefore, further vascular prostheses of pure polymers (Example 1B and Comparative Example 1) and different mixtures of the two (Examples 2B and 3 to 5) were prepared in Examples 1B, 2B and 3 to 5 using identical spinning parameters.

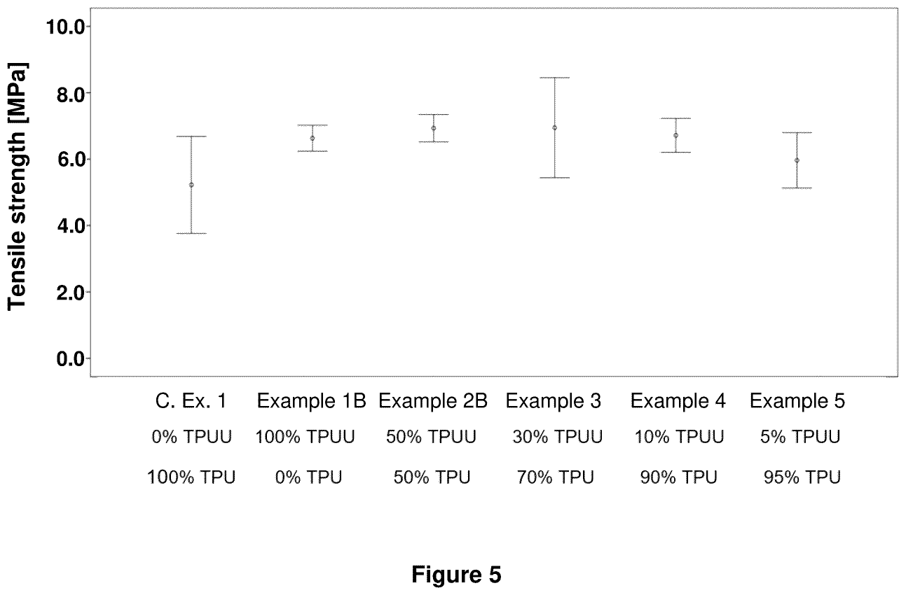

Subsequently, they were each stored for 7 d at room temperature in a physiological saline and then again examined with regard to their tensile strength using the ElectroForce® TestBench by Bose, however, measuring the force standardized with regard to the surface of the rings in megapascal (MPa). The results are graphically depicted in FIG. 5.

It can be seen that the tensile strength of the vascular prosthesis from Comparative Example 1 consisting only of the TPU from Synthesis Example 14 without any self-enhancing effect was, as expected, much lower with 5.2 MPa than that of the prosthesis from Example 1B consisting of pure TPUU of Formula (I), for which a 25% higher value of approximately 6.5 MPa was measured. Considering the sometimes relatively wide standard deviation of the mean values (from 4 to 12 individual determinations), approximately the same tensile strengths were found for the prostheses consisting of the mixtures of the two polymers from Example 2B (TPUU:TPU 50:50), Example 3 (30:70) and Example 4 (10:90) as for the TPUU alone, wherein the mean values for these three examples tend to be even higher than for the prosthesis from Example 2B of TPUU alone. Only at a weight proportion of the self-enhancing TPUU of Formula (I) in the single-digit percentage rage, the prosthesis from Example 5 (TPUU:TPU 5:95), a reduction of tensile strength can be seen.

It can be concluded that even small portions of self-enhancing TPUUs of Formula (I) in mixtures with polymer materials generally used for this purpose have an advantageous effect on the tensile strength of the vascular prostheses made therefrom. And irrespective of whether these materials are TPUs or other polymers used for producing vascular prostheses, such as polyester (e.g., PET), polyolefins (e.g., ePTFE) or the like.

### Degradability Testing

The TPUU obtained in Synthesis Example 1 was dissolved in abs. DMF at a concentration of 10% by weight. This solution was poured into Teflon molds, sized 60×40×2 mm, and the solvent was removed by evaporation at room temperature. After 24 h, the foils thus obtained were dried for further 3 d in the desiccator under vacuum, after which their thickness was measured using an electronic external measuring gauge K110T from Kroeplin, which was about 800 μm. 15 circular disks each with a diameter of 5 mm were die-cut from this foil, and their exact weight was determined, which was between 15 and 20 mg in every case.

Subsequently, one disk each was put in a test tube with 20 ml of PBS (1X, pH 7.4) as simulation of physiological conditions, whereafter the test tubes were heated in an autoclave at 90° C. After 7, 14, 25, 35 and 41 d, respectively, three were taken out. The disks contained therein were each put into deionized water for three times 15 min each, in order to remove the salts contained therein. Then, the drained weight and—after drying to a constant weight (24 h at 80° C. and 120 mbar)—the dry weight were determined and a molecular weight determination via gel permeation chromatography was conducted. From the values thus, mass loss, molecular weight reduction, and swelling of individual samples were calculated based on the following equations 1 to 3.

$${m}_{eros} ( t ) = \frac{{m}_{t} - {m}_{0}}{{m}_{0}} \cdot 100 \qquad Equation 1$$

$$\% {\overline{M}}_{w} ( t ) = \frac{{\overline{M}}_{w} ( t )}{{\overline{M}}_{w} ( 0 )} \cdot 100 \qquad Equation 2$$

$$s ( t ) = \frac{{m}_{t}^{w} - {m}_{t}}{{m}_{t}} \cdot 100 \qquad Equation 3$$

mₑᵣₒₛ(t) Mass loss after t days degradation
t degradation time in d
mₜ Sample weight after t days degradation in mg
m₀ Sample weight before degradation in mg
% M̅_{w}(t) Molecular weight reduction after t days degradation in %
M̅_{w}(t) Molecular weight after t days degradation in kDa
M̅_{w}(0) Molecular weight before degradation in kDa
s(t) Swelling of the sample after t days degradation in %
mₜ^{w} Drained weight of the sample after t days degradation in mg

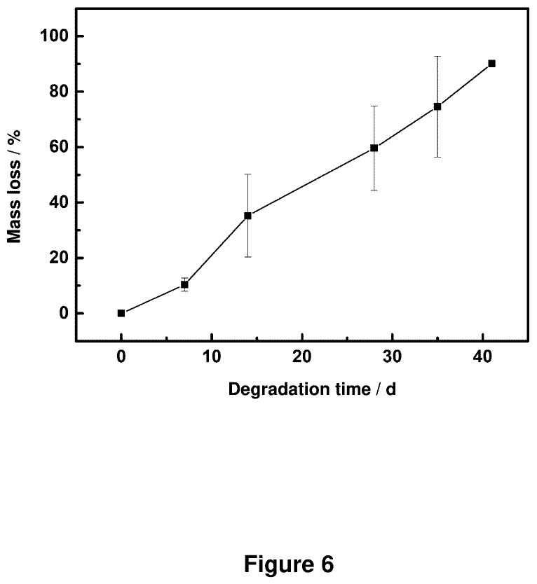

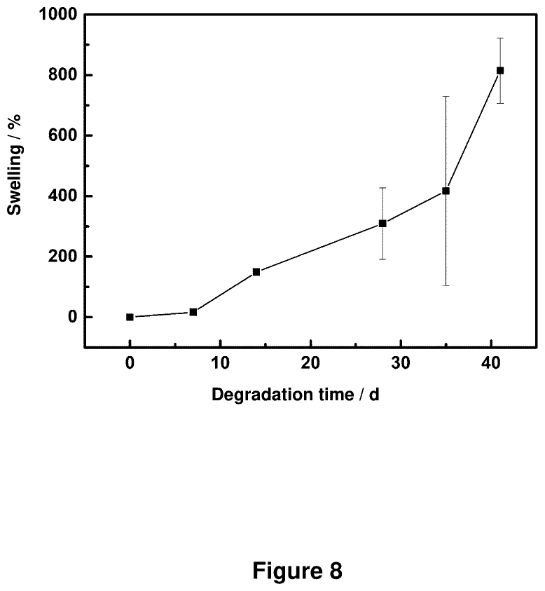

The results of this calculation are graphically depicted in FIGS. 6 to 8.

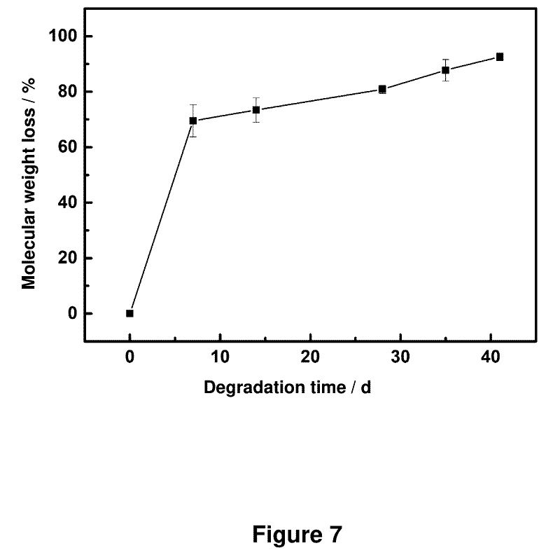

This mainly shows that already after 7 d, a molecular weight reduction of approximately ¾ of the starting molecular weight had occurred—with a mass loss of approximately 10% and hardly any swellability, which gradually increased to approximately 90% until the end of the degradation study after 41 d—just like the mass loss. An extrapolation of the graphs of FIGS. 6 and 7 leads to the conclusion that the degradation was probably complete after another week at 90° C. PBS, i.e., after 7 weeks in total.

The TPUU of Formula (I) used for preparing vascular prostheses according to the present invention can thus be completely hydrolytically degraded within a few weeks and in this aspect excellently suited for this purpose.

### In Vitro Biocompatibility Test

### A) Preparation of Macrophage, EPC and HUVEC Cultures

For the culture of primary macrophages and endothelial precursor cells (EPCs), freshly donated blood from healthy human donators (45 ml) was separated via density centrifugation with Ficoll Paque (GE Healthcare, USA) for 30 min at 300 g without braking. The buffy coat with peripheral mononuclear blood cells (PBMCs) was carefully pipetted into a fresh centrifuge tube, filled up to 50 ml with phosphate-buffered silane (PBS, Sigma-Aldrich) and spun for 10 min at 500 g in order to wash the cells and to remove thrombocytes. This step was repeated once. The EPCs were then used for population experiments of the rings with a width of 2 mm. In order to differentiate macrophages, the cells were resuspended in a RPMI 1640 medium (supplemented with 10% FBS, Sigma-Aldrich, USA), counted and seeded in T175 cell culture bottles (50×10⁶ cells per bottle) for 2 h in an incubator at 37° C. After 2 h, the cell culture medium was removed, the cells were carefully washed twice with PBS, and RPMI 1640 with 50 ng/ml M-CSF (macrophage-colony stimulating factor) was added. After 3 d, another exchange of medium was conducted with M-CSF, and the cells were used for experiments on the next day.

Human umbilical vein endothelial cells (HUVEC, pooled donator, Lonza, Switzerland) were cultivated in endothelial growth medium (EGM-2, Lonza, Switzerland) supplemented with 10% of FBS. The exchange of medium was conducted every other day. The cells were used for the experiments between passage 3 and 6.

B) Cytotoxicity Tests with HUVEC and Macrophage Cultures

XTT (2,3-bis(2-methoxy-4-nitro-5-sulfophenyl)-5-[(phenylamino)carbonyl)-2H-tetrazoliumhydroxide) can be reduced to a formazan product in viable cells. The result can be determined by photometric measurements and correlated with the number of viable cells. The assay was conducted based on ISO10993-5.

### B1—Comparison of Polymers

As described above, foils were drawn from the TPUU obtained in Synthesis Example 1, from which circular disks with a diameter of 5 mm were die-cut (“TPUU”). For comparison, disks were prepared in the same way from the TPU obtained in Synthesis Example 14 (“TPU1”), from Pellethane™ 2363-80A, a commercially available TPU from Lubrizol Inc. of methane-4,4′-diphenyldiisocyanate (MDI), polytetrahydrofuran (pTHF), and 1,4-butanediol (BDO) (“TPU2”), as well as a TPU described in Ehrmann et al. (2020; supra), namely a polycarbonate urethane of polyhexamethylenecarbonate (pHMC), hexamethylenediisocyanate (HMDI) and bis(3-hydroxypropylene)carbonate (BHPC) (“TPU3”). These three TPUs have already shown to be suitable for the syntheses of vascular prostheses in the past.

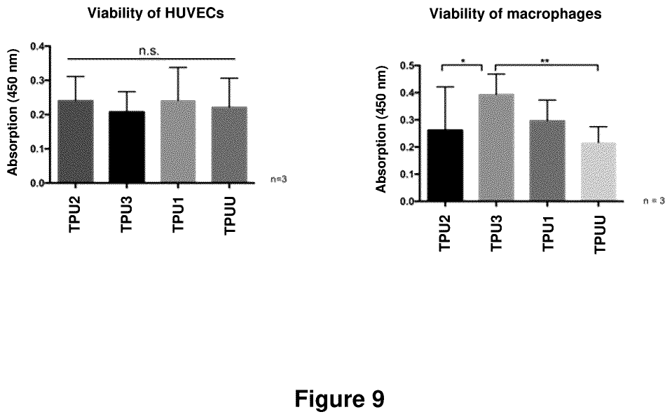

HUVECs or macrophages were seeded on the polymer disks at a cell number of 10,000 cells/disk in a 96 well plate for 24 h until a confluent cell layer was formed. XTT powder was dissolved in a cell culture medium with 60° C. at a concentration of 1 mg/ml. PMS (phenazine methosulfate, Sigma-Aldrich, USA) was dissolved in PBS at a concentration of 5 mM. For preparing a working solution, which was added to cells, PMS was pipetted into the XTT solution at a concentration of 25 μM. The cells were incubated with 50 μl of this XTT working solution for 4 h at 37° C. in a cell culture incubator. The medium was then transferred to a new 96 well plate, and absorption at 450 nm was measured with a reference wavelength of 620 nm. The results are shown in FIG. 9 as mean values of three determinations.

The graphs show that for the four examined polymers, there is not too much of a difference concerning viability of HUVECs and macrophages. In the case of the macrophage culture, the TPUU from Synthesis Example 1 gave the worst results, however, cytotoxicity of all four examined polymers was in the same order of magnitude, which again proves suitability of the TPUU for preparing vascular prostheses.

### B2—Monomer Test

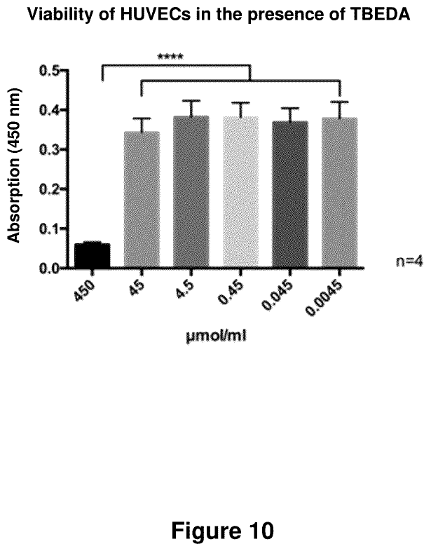

In order to assess the cytotoxicity of the monomer N,N′-di-tert-butylethylenediamine (TBEDA) contained in the TPUU, which was used for preparing vascular prostheses for the first time in the examples, HUVECs were seeded in 96 well plates and cultivated until a confluence of 80% was reached. TBEDA was added in the form of a solution in dimethyl sulfoxide (DMSO) so that a concentration of 450 μmol/ml was achieved, from which a dilution series with a dilution factor of 10 was then created. The solutions were then all incubated for 24 h, whereafter the XTT test was conducted as described above, the results of which are shown as mean values of quadruple determinations in FIG. 10. It can be seen that the viability of HUVECS was already at an acceptable level from a TBEDA concentration of 45 μmol/ml.

### C) Adhesion and Proliferation of Human Endothelial Precursor Cells

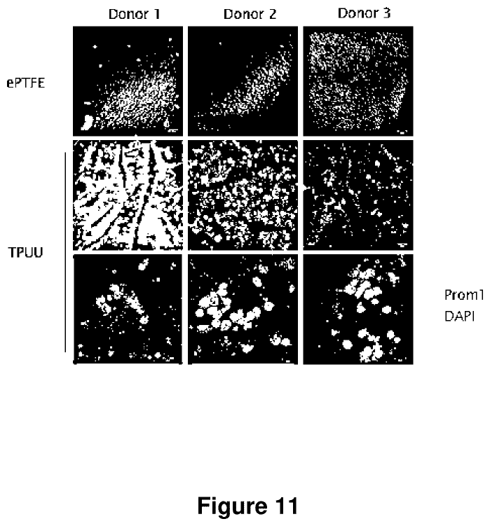

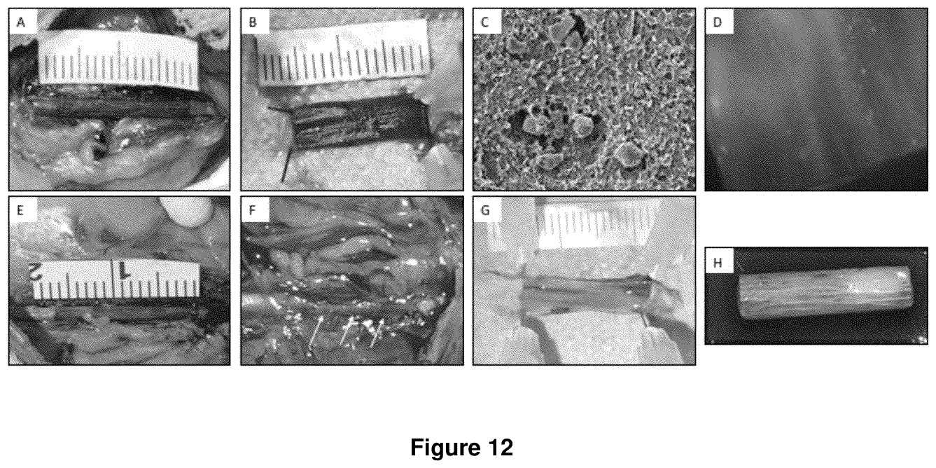

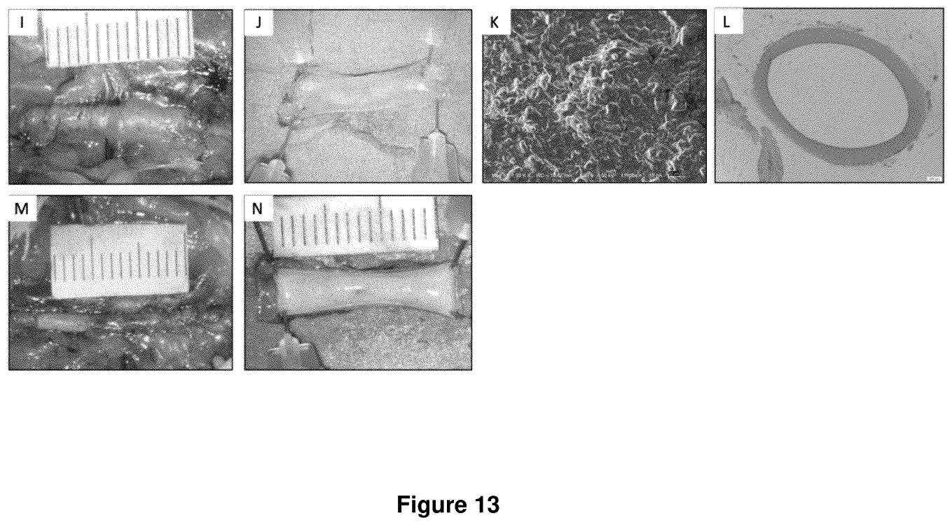

As described above, PBMCs were isolated from fresh human blood from three different donators and populated at a cell number of 2 mil cells per ml on rings with a width of 2 mm of the TPUU electrospun in Example 1A as well as on expanded polytetrafluoroethylene (ePTFE) as control. After two weeks of incubation with three medium exchanges per week, the cells were fixed over night with 4% paraformaldehyde and then, after several washings with PBS, died with the EPC-specific antibody against Pro1 (Invitrogen, USA, Cat. No.: MA1-219) for 45 min at room temperature. After further two washing steps with PBS, they were incubated with the secondary conjugated antibody Alexa Fluor 647 (Abcam, Cat. No: ab150115), again for 45 min at room temperature. After a final washing step with PBS, the samples were applied to microscope slides with the aid of a cover medium, and photographs of the cells were taken with a LSM-700 confocal microscope (Zeiss, Germany). These are shown in FIG. 11.

It can be seen that the number of adhered EPCs on the prosthesis from Example 1A is considerably increased compared to ePTFE, even though there are relatively big differences between the different donators concerning Prom1 expression and the number of adhered cells. The TPUU used in Example 1A was in any case populated by EPCs at a significantly higher level that the control, which underlines its suitability for preparing vascular prostheses.

### In Vivo Biocompatibility Tests

Animal experiments were approved by the Animal Experiments Commission of the Medical University of Vienna and the Austrian Federal Ministry of Science and Research. Vascular prostheses electrospun in Example 2A (ID: 1.5 mm, length: 20 mm), as shown in FIG. 2a, were implanted microsurgically into the infrarenal aorta of twenty-one inbred rats of the Sprague Dawley species (male, body weight 300-400 g) (end-to-end anastomoses, suture material: nylon 9/0). They received neither anticoagulation nor thrombocyte aggregation inhibitors. The implants were removed after 2 h, 7 d, 3 months or 6 months under general anesthesia with full heparinization of the animals and macroscopically examined. Parts of the proximal and distal anastomosis region and the graft center were preserved either in formalin or glutaraldehyde, 2.5%, and then histologically examined through hematoxylin staining, immunohistochemistry or scanning electron microscopy.

Furthermore, the thrombocyte adhesion was examined through mepacrine staining. Here, the transplant pieces were, after explanation, stored for 24 h at 4° C. in 2.5% glutaraldehyde. The samples were washed three times for 1 min in PBS and then incubated in a mepacrine staining solution (10 mM in distilled water, Sigma-Aldrich, USA) for 90 min at room temperature in the dark. The samples were washed 3 times in PBS to remove residual staining solution, and embedded on glass slides with fluorescent embedding medium. Adhering thrombocytes were observed with an inverse microscope by Olympus and recorded.

The following photographs are shown in FIGS. 12A-H:

FIG. 12A shows the vascular prosthesis, and FIG. 12B shows the luminal surface of the prosthesis after an implantation time of 2 hours;

FIG. 12C shows an electron microscope image of the luminal surface;

FIG. 12D shows mepacrine staining of the adhered blood platelets after an incubation time of 2 hours, with almost no visible adhered thrombocytes;

FIG. 12E shows the prosthesis directly after implantation, and FIG. 12F shows the prosthesis after an implantation time of seven days;

FIG. 12G shows the luminal surface after an implantation time of seven days, with no visibly thromboses; and

FIG. 12H shows the prepared prosthesis after an implantation time of one week, with no signs of premature degradation and no visible defects or aneurysms.

And FIGS. 13I-N show the following photographs:

FIG. 13I shows the vascular prosthesis, FIG. 13J shows its luminal surface, FIG. 13K shows an electron microscopic image of the luminal surface, and FIG. 13L shows a histological section of the prosthesis, all after an implantation time of 3 months, with no visible adhered thrombocytes; and

FIG. 13M shows the prosthesis and FIG. 13N its luminal surface after an implantation time of 6 months, after which no thromboses are visible.

The tubes electrospun in Example 2A of the invention are consequently excellently suitable for use as vascular prosthesis.

### Tests for Self-Enhancing Properties

In analogy to the tests described above concerning the degradability of the prosthesis material, a number of foils were drawn from 10% DMF solutions of the polymers obtained in Synthesis Examples 1 to 16, i.e., the TPUUs of Formula (I) from Synthesis Examples 1 to 13, the TPU from Synthesis Example 14, and the TPUUs of Synthesis Examples 15 and 16 without sterically hindered amino groups in the sense of the invention, and also from a solution of the Pellethane® 2363-80A TPU commercially available from Lubrizol LifeSciences.

Pellethane® 2363-80A is a thermoplastic polyurethane without urea moieties prepared from methylenedi(phenylisocyanate) (MDI), polytetrahydrofuran (pTHF) and 1,4-butanediol, which is not biodegradable and has a molecular weight Mₙ of approximately 37 kDa and a M_{w} of approximately 63 kDa.

The foils were, for the indicated duration of time (24 h, 7 d or 28 d), dry- or wet-stored (and dried), whereafter three parts of the foils each were die-cut as type 5B tensile testing samples and subjected to tensile testing according to ISO 527-1 using a Zwick Z050 tensile tester, wherein the samples that had been chucked in the tensile tester were pulled apart at a speed of 50 mm/min until they broke. Each sample was tested in triplicate, the results were averaged, and the thus measured elongation at break (as a percentage of the initial length) was used as a measure for the foil tear strength and the ultimate tensile strength (in MPa) as a measure for tensile strength. Table 1 below shows direct comparisons of the mean values thus obtained for every time as difference between mean values that were calculated for the wet-stored samples and the dry-stored samples, Δ_{wet-dry}, each indicated as a percentage of the value for the dry sample.

Table 1 overleaf lists, as mentioned before, the differences of the mean values of the values that were measured for all samples of sterically hindered TPUUs of Formula (I) of Synthesis Examples 1 to 13 (“ster.hind.”), the two TPUUs of Synthesis Example 15 and 16 without steric hindrance (“w.o. ster.h.”) and the two TPUs also not sterically hindered (Synthesis Example 14 and Pellethane® 2363-80A) after their respective storage times (24 h, 7 d, or 28 d) at room temperature. Since, in most cases, the values that were determined after 7 d were already representative, only the value after 7 d was determined for some later foil samples.

Due to the fact that the amount of sterical hindrance of the secondary diamines that were employed as chain extenders for introducing the radical C₁ was small to nonexistent with the TPUUs of Synthesis Examples 12 and 13 as well as Synthesis Examples 15 and 16, those four TPUUs were subjected another test series at 60° C. to enhance their reactivity. The respective differences of the mean values after a storage time of 24 h as well as 7 d are indicated in Table 2 below.

TABLE 1
Results of tensile testing with three foils each, wet-
stored or dry-stored, respectively, at room temperature

| | | Tear strength, [%] | | | Tensile strength, [MPa] | | |
|---|---|---|---|---|---|---|---|
| | | Δ_{wet-dry} [%] | | | Δ_{wet-dry} [%] | | |
| Example | Polymer | 24 h | 7 d | 28 d | 24 h | 7 d | 28 d |
| Synthesis Example 1 | ster. hind. TPUU | −6 | 30 | −14 | −3 | 67 | 30 |
| Synthesis Example 2 | ster. hind. TPUU | −4 | 10 | 43 | −1 | 48 | 17 |
| Synthesis Example 3 | ster. hind. TPUU | 4 | 1147 | 425 | 0 | 331 | 107 |
| Synthesis Example 4 | ster. hind. TPUU | −9 ¹⁾ | −9 | 12 ¹⁾ | 15 ¹⁾ | 23 | 82 |
| Synthesis Example 5 | ster. hind. TPUU | 74 | 176 | 625 | 45 | 16 | 52 |
| Synthesis Example 6 | ster. hind. TPUU | −1 ¹⁾ | 5 ¹⁾ | 24 | 35 | 51 | 81 |
| Synthesis Example 7 | ster. hind. TPUU | −80 | −30 | −28 ¹⁾ | 55 | 33 | 22 ¹⁾ |
| Synthesis Example 8 | ster. hind. TPUU | 136 | 288 | 367 | 133 | 297 | 286 |
| Synthesis Example 9 | ster. hind. TPUU | 317 | 818 | 1688 | 38 | 75 | 552 |
| Synthesis Example 10 | ster. hind. TPUU | 0 ¹⁾ | 0 | 44 ¹⁾ | 0 ¹⁾ | 0 ¹⁾ | 32 |
| Synthesis Example 11 | ster. hind. TPUU | 226 | 306 | 548 | 105 | 85 | 103 |
| Synthesis Example 12 | ster. hind. TPUU | −14 ¹⁾ | −1 ¹⁾ | 1 ¹⁾ | 0 ¹⁾ | 11 ¹⁾ | 4 ¹⁾ |
| Synthesis Example 13 | ster. hind. TPUU | | −4 | | | 54 | |
| Synthesis Example 14 | TPU | −1 ¹⁾ | 22 ¹⁾ | 9 ¹⁾ | 7 ¹⁾ | −1 ¹⁾ | 4 ¹⁾ |
| Pellethane ® | TPU | −3 ¹⁾ | 1 ¹⁾ | 7 ¹⁾ | 2 ¹⁾ | 14 ¹⁾ | 1 ¹⁾ |
| Synthesis Example 15 | TPUU w.o..ster.h. | | −7 ¹⁾ | | | 10 ¹⁾ | |
| Synthesis Example 16 | TPUU w.o..ster.h. | | −5 ¹⁾ | | | 9 ¹⁾ | |

¹⁾ statistically not significant, since the standard deviations of the mean values overlap

TABLE 2
Results of tensile testing with three foils each,
wet-stored or dry-stored, respectively, at 60° C.

| | | Tear strength, [%] | | Tensile strength, [MPa] | |
|---|---|---|---|---|---|
| | | Δ_{wet-dry} [%] | | Δ_{wet-dry} [%] | |
| Example | Polymer | 24 h | 7 d | 24 h | 7 d |
| Synthesis Example 12 | ster. hind. TPUU | 5 ¹⁾ | 9 ¹⁾ | 52 | 146 |
| Synthesis Example 13 | ster. hind. TPUU | −13 | | 76 | |
| Synthesis Example 15 | TPUU w.o. ster. h. | −30 ¹⁾ | −21 ¹⁾ | −18 ¹⁾ | −52 ¹⁾ |
| Synthesis Example 16 | TPUU w.o. ster. h. | 2 ¹⁾ | −15 ¹⁾ | 28 ¹⁾ | 3 ¹⁾ |

¹⁾ statistically not significant, since the standard deviations of the mean values overlap

It is clear from Table 1 that for the sterically hindered TPUUs of Formula (I) from Synthesis Examples 1 to 13 for the majority of the measured values a contact with water at room temperature led to an improvement of the tear strength or tensile strength, respectively, by a double-digit percentage range (highlighted in bold). In the non-sterically-hindered TPUUs or TPUs, this is only the case with three measured values which also show overlapping standard deviations of mean values and are therefore not statistically significant. Therefore, as expected, no significant change in mechanical properties after a wet storage was measurable in any samples of the sterically hindered polymers.

As a measure for “self-enhancement” of the solid samples prepared from the sterically hindered TPUUs of Formula (I) due to recombination reactions when contacted with water, as shown in Scheme D (or for the chain extender only sterically hindered on one side of Synthesis Example 11 in Scheme E, respectively), especially the tensile strength improvements are relevant. Here, six out of twelve TPUUs of Formula (I) already show an improvement in a double-digit percentage range after 24 h and even eleven out of thirteen TPUUs of Formula (I) after 7 d or 28 d of wet storage, respectively. Furthermore, for four cases all measured values were substantially improved after a wet storage compared to a dry storage, among them also the TPUU from Synthesis Example 11 having only one unstable urea group per C₁ unit.

Consequently, the occurrence of the above mentioned recombination reactions was demonstrated for almost all TPUUs of Formula (I)—representing various components and different proportions of sterically hindered urea groups per molecule. The only exception being the TPUU of Synthesis Example 12 with nitrogen atoms substituted with isopropyl that caused a relatively low sterical hindrance.

Therefore, the TPUU of Formula (I) from Synthesis Example 12 as well as the one from Synthesis Example 13, using 2,6-dimethylpiperazine as the diamine sterically hindered on one side, together with both non-sterically hindered piperazine containing TPUUs of Synthesis Examples 15 and 16, i.e., with piperazine or 2,5-dimethylpiperazine, respectively, as C₁ units, were subjected to another test at 60° C., to determine whether the reactivity of the urea group could be increased with higher temperatures.

As apparent from Table 2, this was the case for both sterically hindered TPUUs of Formula (I), especially since the tensile strength for the isopropyl-containing polymer of Synthesis Example 12 was already increased after 24 h by more than 50% and after 7 d by almost 150%. The 2,6-dimethylpiperazine-containing TPUU of Synthesis Example 13 also showed an increased tensile strength by over 75% after already 24 h at 60° C. In contrast, the increase in temperature in the non-sterically hindered TPUUs of Synthesis Example 15 and 16 containing piperazine or 2,5-dimethylpiperazine, respectively, did not have the desired effect—on the contrary: in this case, the values at 60° C. were even worse than after storage at room temperature.

This clearly shows that the piperazine-containing or 2,5-dimethylpiperazine-containing TPUUs, respectively, from the literature cited in the beginning are not sterically hindered according to the invention, whereas TPUUs of Formula (I) substituted with isopropyl certainly are.

Comparatively bad were also the results at room temperature for the TPUU of Synthesis Example 7 in which the tear strength compared to dry storage even decreased and a clear improvement of the tensile strength after only 24 h weakened in the course of further wet storage, as well as for the one of Synthesis Example 10 in which a clear improvement was observed only after 28 d. While not wishing to be bound by theory, the inventors attribute this to the following circumstances.

The self-enhancing effect caused by the recombination reactions is not only influenced by the type and position of the unstable urea groups in the molecule, but also strongly by the structure of the polymer, i.e., the relative self-enhancement depends on the storage time as well as on the building blocks of the TPUU. Specifically, the stable urea groups formed by the recombination reactions result in a stiffening of the polymer matrix as a whole. At a certain concentration of these urea groups, this leads to the self-enhancing effect being “saturated”. Consequently, sterically hindered, unstable urea groups that still exist at this point cannot undergo a recombination reaction according to the principle of Schemes D and E since the primary amines and isocyanates formed in situ in the matrix are no longer mobile enough to bond with each other. As a result, from this point on, they no longer serve as self-enhancing groups, but rather as degradable groups, as shown in Scheme F. Because of this, especially when stored in water for a longer period of time and/or in case of a high number of sterically hindered urea groups, the self-enhancing effect can be weakened or even turn into a degradation effect.

This effect is especially pronounced for the TPUU of Synthesis Example 7 that has a very rigid matrix due to the presence of H12MDI and pHMC. Even though a pronounced self-enhancement was observed after only 24 h it had already decreased in test samples that were wet-stored for 7 d. After 28 d of wet storage no significant self-enhancing effect caused by newly formed stable urea groups was observed since it was compensated for by the degradation of still existing sterically hindered urea groups.

The reason why for the TPUU of Formula (I) of Synthesis Example 10 improvements could only be observed after 28 d could be attributed to the fact that a hydrolysis of part of the lactic acid ester bonds compensated for the self-enhancing effect achieved by recombination, i.e., again degradation reactions. When using the TPUUs of Formula (I) as temporary body implants, e.g., vascular prostheses, such a hydrolysis may be desirable, though.

In any case, this self-enhancement's dependence on the matrix stiffness induced by the remaining components (macrodiol, diisocyanate, chain extenders) and on simultaneously occurring degradation reactions shows that the ideal wet storage time for achieving the respective desired effect obviously varies for differently composed TPUUs of Formula (I).

However, the above experiments also clearly show that the composition of the TPUUs of Formula (I) is variable within very broad limits without losing properties relevant for their suitability for the preparation of temporary body implants such as vascular prostheses. These include, in particular, the self-enhancing effect of the prostheses occurring after implantation due to the presence of the sterically hindered amino group in the radicals C₁—for which only two amino groups substituted with the low-bulky isopropyl radical (see Synthesis Example 12) or only one tert-butyl-substituted amino group (see Synthesis Example 11) per C₁ radical are sufficient, but also the simultaneous biodegradability of the TPUUs of Formula (I) due to the presence of the ester moieties in one or more of the radicals I, M, C₁ and C₂ cleavable under physiological conditions.

Also the fact that the vascular prosthesis prepared in Example 1A exclusively from a TPUU of Formula (I) delivered poorer values in terms of porosity than the prosthesis from Example 2A, where the same TPUU was electrospun in a mixture with 50% by weight of a conventional TPU, does not reduce the basic suitability of such TPUUs for the preparation of vascular prostheses. On the one hand, the test results shown in FIG. 5 prove that the tensile strengths of mixtures of TPUU and TPU measured after wet storage exceed those of a conventional TPU alone, even when only very small amounts of the new TPUU are added to the TPU. And secondly, it may be assumed that TPUUs of Formula (I) with different compositions will give better porosity values than the TPU used in Example 1A, e.g., TPUUs which also have bulky substituents or side chains in the radicals I, M and/or C₂. The definitions of the maximum number of carbon atoms of the radicals I, C₁ and C₂ or the molecular weight of M offer a sufficient degree of leeway in this respect.

In any case, it is clearly demonstrated herein that the thermoplastic poly(urethane-urea) polyadducts of Formula (I) with sterically hindered urea groups can, in a solid state, by treating them with water, be converted to new polymers, the physical characteristics of which are improved in many ways compared to those of the starting polymers. Therefore and due to their physiological degradability, the TPUUs of Formula (I) are extremely well suited for producing vascular prosthesis be electrospinning according to the present invention.

1.-15. (canceled)

16. A method for producing vascular prostheses by electrospinning a thermoplastic poly(urethane-urea) polyadduct with sterically hindered urea groups of the following Formula (I): 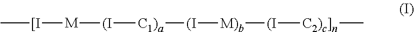
wherein I, M, C₁ and C₂ each represent bivalent radicals that are linked to each other via a urethane or urea moiety, whereof
each I independently represents a bivalent, saturated or unsaturated, aliphatic, alicyclic or aromatic radical with 1 to 20 carbon atoms derived from a diisocyanate;
each M independently represents a bivalent radical of an aliphatic polyether, polyester or polycarbonate derived from a macrodiol having a number average molecular weight Mₙ≥500;
each C₁ independently represents a bivalent, saturated or unsaturated, aliphatic or alicyclic radical with 1 to 30 carbon atoms derived from a diamine or amino alcohol with at least one sterically hindered secondary amino group through removal of one N-linked hydrogen atom each of the diamine or one N-linked and the O-linked hydrogen atoms of the amino alcohol;
each C₂ independently represents a bivalent, saturated or unsaturated, aliphatic, alicyclic or aromatic radical with 1 to 20 carbon atoms derived from a diol, diamine or amino alcohol without sterically hindered secondary amino groups;
wherein, in the radicals I, C₁ and C₂, when more than four carbon atoms are present, optionally at least one of them is substituted by a heteroatom selected from oxygen and nitrogen;
wherein at least one of the radicals I, M, C₁ and C₂ comprises one or more ester moieties; and
a, b and c each independently represent an integer from 0 to 10, and n is a number≥3 representing the number of blocks in the polyadduct;
provided that within each separate block a+c≥1 and in all blocks together at least one a≥1 and at least one c≥1.

17. The method according to claim 16, wherein in the thermoplastic poly(urethane-urea) polyadduct at least part of the ester moieties is cleavable under physiological conditions, that the radicals I, M, C₁ and C₂ as well as any cleavage products thereof are biocompatible and physiologically acceptable, and a temporary vascular prosthesis is prepared by the electrospinning method.

18. The method according to claim 16, wherein in the thermoplastic poly(urethane-urea) polyadduct:
a and c are each independently ≤5 or ≤3; and/or
a and c are each independently ≥1; and/or
b≥1; and/or
b=c or b=a or b+1=a+c; and/or
n≥5 or n≥10 or n≥50.

19. The method according to claim 16, wherein in the thermoplastic poly(urethane-urea) polyadduct:
the radicals I are each independently derived from a diisocyanate selected from the following group: 1,6-hexamethylene diisocyanate, 4,4′-diisocyanatodicyclohexylmethane, isophorone diisocyanate, 1,3-bis(isocyanatomethyl)cyclohexane, diphenylmethane-4,4′-diisocyanate, L-lysine ethyl ester diisocyanate; and or
the radicals M are each independently derived from a polyether, polyester or polycarbonate selected from the following group: polytetrahydrofuran, polyethylene glycol, polypropylene glycol, polycaprolactone, polylactide, polyglycolide, poly(lactide-co-glycolide), polyhexamethylene carbonate.

20. The method according to claim 16, wherein in the thermoplastic poly(urethane-urea) polyadduct, the radicals C₁ are each independently derived from a diamine and selected from radicals of the following Formula (II): 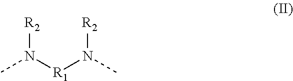
wherein the dashed lines each show the linkage to the carbonyl group of a urethane or urea moiety linking the radicals I, M, C₁ and C₂,
R₁ is selected form bivalent, saturated or unsaturated, aliphatic or alicyclic radicals with 1 to 20 carbon atoms; and
the R₂ are each independently selected from hydrogen and monovalent, bulky, saturated or unsaturated, aliphatic or alicyclic radicals with 1 to 10 carbon atoms, provided that not both R₂ are simultaneously hydrogen.

21. The method according to claim 20, wherein in the thermoplastic poly(urethane-urea) polyadduct:
R₁ is selected from C₁-C₁₀-alkylene or C₄-C₁₀-cycloalkylene radicals or from C₂-C₆-alkylene and C₅-C₆-cycloalkylene radicals; and/or
the R₂ are each independently selected from 1,1-dimethyl-substituted, saturated or unsaturated C₁-C₆-alkyl radicals or 1-methyl-substituted C₃-C₆-cycloalkyl radicals or from isopropyl, tert-butyl, 1,1-dimethylpropyl and 1-methylcyclohexyl.

22. The method according to claim 16, wherein in the thermoplastic poly(urethane-urea) polyadduct at least one of the radicals C₂ comprises one or more ester moieties, which are optionally each independently derived from a diol from the following group: bis(hydroxyethyl) terephthalate, bis(hydroxypropyl) carbonate, 2-hydroxyethyl lactate, and 2-hydroxyethyl glycolate.

23. The method according to claim 16, wherein in the thermoplastic poly(urethane-urea) polyadduct b+1=a+c and the polyadduct corresponds to the following Formula (IV): 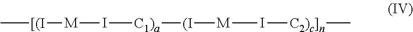
wherein
a and c are each independently selected from 1 to 3 or
a and c are each 1; and
n≥5 or n≥10 or n≥20.

24. The method according to claim 16, wherein in the electrospinning method, a solution of the TPUU of Formula (I) in an organic solvent or a solvent mixture in an electrospinning device that comprises a high-voltage generator, a syringe pump, a syringe with a blunt end as an electrode, a grounded, electrically conductive rotating steel mandrel as a collector electrode, and optionally an auxiliary electrode, is injected by means of the syringe into the electric field built up between the electrodes, and the polymer fibers that are formed as continuous nanofibers are wound onto the rotating mandrel as a tube suitable as a vascular prosthesis.

25. The method according to claim 24, wherein a solution of the TPUU of Formula (I) in hexafluoroisopropanol is used.

26. The method according to claim 24, wherein a solution of a mixture of the TPUU of Formula (I) and at least one further polymer is used and that tubes consisting of the mixture are prepared.

27. The method according to claim 26, wherein the at least one further polymer is used in a proportion of at least 10% by weight, at least 30% by weight or at least 50% by weight of the mixture.

28. The method according to claim 26, wherein a mixture of the TPUU of Formula (I) and one biodegradable TPU is used.

29. The method according to claim 28, wherein a polyether urethane, such as a polyadduct of polytetrahydrofuran, bis(hydroxyethyl) terephthalate and hexamethylene diisocyanate, is used as the biodegradable TPU in the mixture.

30. A vascular prosthesis obtained by the electrospinning method using a thermoplastic poly(urethane-urea) polyadduct of Formula (I) according to claim 16.
